SEQUENCE LISTING (23 sequences)

SEQ ID NO: 1 - protein, 10 aa, synthetic construct
SASSSVSYMN

SEQ ID NO: 2 - protein, 6 aa, synthetic construct
DTSKLA

SEQ ID NO: 3 - protein, 9 aa, synthetic construct
QQWSSNPFT

SEQ ID NO: 4 - protein, 5 aa, synthetic construct
RYTMH

SEQ ID NO: 5 - protein, 17 aa, synthetic construct
YINPSRGYTNYNQKVKD

SEQ ID NO: 6 - protein, 8 aa, synthetic construct
YYDDHYCL

SEQ ID NO: 7 - protein, 23 aa, synthetic construct
AATMETDTLLLWVLLLWVPGSTG

SEQ ID NO: 8 - protein, 25 aa, synthetic construct
LAATMKCSWVIFFLMAVVTGVNSQV

SEQ ID NO: 9 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 10 - protein, 115 aa, synthetic construct
QLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTPVTV

SEQ ID NO: 11 - protein, 57 aa, synthetic construct
ANESHNGSILPISWRWGESDMTFICVARNPVSRNFSSPILARKLCEGAADDPDSSMV

SEQ ID NO: 12 - protein, 214 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 449 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 107 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 15 - protein, 11 aa, synthetic construct
KASQDVGIAVA

SEQ ID NO: 16 - protein, 7 aa, synthetic construct
WASTRHT

SEQ ID NO: 17 - protein, 9 aa, synthetic construct
QQYSSYPYT

SEQ ID NO: 18 - protein, 119 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 19 - protein, 5 aa, synthetic construct
RYWMS

SEQ ID NO: 20 - protein, 17 aa, synthetic construct
EINPDSSTINYAPSLKD

SEQ ID NO: 21 - protein, 10 aa, synthetic construct
PDGNYWYFDV

SEQ ID NO: 22 - protein, 130 aa, synthetic construct
AATMETDTLLLWVLLLWVPGSTGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 23 - protein, 140 aa, synthetic construct
LAATMKCSWVIFFLMAVVTGVNSQVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTPVTV

(19) United States
(12) Patent Application Publication
(10) Pub. No.: US 2025/0074982 A1
(43) Pub. Date: Mar. 6, 2025

(54) BISPECIFIC ANTIBODIES AND USES THEREOF

(71) Applicant: City of Hope, Duarte, CA (US)

(72) Inventors: Xiuli Wang, Temple City, CA (US); John Ernest Shively, Arcadia, CA (US); Dennis Awuah, Sierra Madre, CA (US); Maciej Kujawski, Azusa, CA (US); Lin Li, Monrovia, CA (US); Paul Yazaki, Glendale, CA (US)

(21) Appl. No.: 18/818,983

(22) Filed: Aug. 29, 2024

Related U.S. Application Data
(60) Provisional application No. 63/535,166, filed on Aug. 29, 2023.

(51) Int. Cl. C07K 16/28; A61K 39/00; A61P 35/00

(52) U.S. Cl. CPC C07K 16/2809; A61K 40/11; A61K 40/33; A61K 40/421; A61P 35/00; C07K 16/2803; A61K 2239/48; C07K 2317/24; C07K 2317/31; C07K 2317/53; C07K 2317/73

(57) ABSTRACT

Provided herein are, inter alia, antibody compounds comprising an anti-immune cell antibody (e.g., anti-CD3 antibody) covalently bound to a CS-1-binding antibody; immune cells bound to compounds comprising an anti-immune cell antibody (e.g., anti-CD3 antibody) covalently bound to a CS-1-binding antibody; humanized OKT3 antibodies; pharmaceutical compositions; and methods for treating cancer, such as multiple myeloma.

### CROSS-REFERENCE TO RELATED APPLICATION

This application claims the benefit of priority to U.S. Application No. 63/535,166 filed Aug. 29, 2023, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under Grant No. CA238429-01 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing, written in an XML file entitled “Sequence048440-833001US.xml” was created on 5 Aug. 2024 and has a size of 22,292 bytes, is incorporated by reference herein.

### BACKGROUND

Bispecific T cell engaging antibodies (BiTE) that redirect human T cells towards antigen-specific tumor killing, have become a rapidly growing and promising area in cancer immunotherapy. Traditionally, BiTEs consist of engineered single chain variable fragments (scFv) of two antibodies fused by a flexible linker, and although they possess several advantages including availability and dosing regimen, they are subject to rapid kidney clearance because of their small molecular weight. Use of BiTEs therefore requires continuous antibody infusions. Hence, there is a need for developing therapeutics as an alternative to infusion of large quantities of BiTE antibodies. Provided herein are solutions to these and other problems in the art.

### BRIEF SUMMARY

Provided herein are antibody compounds comprising an anti-immune cell antibody covalently bound to an anticancer antibody, such as a CS-1-binding antibody. In embodiments, the CS-1 binding antibody is elotuzumab and the anti-immune cell antibody is an OKT3 antibody. Provided herein are methods of treating cancer, such as multiple myeloma, in a patient in need thereof by administering to the patient an effective amount of the antibody compounds described herein. These and other embodiments are described in detail herein.

### BRIEF DESCRIPTION OF THE DRAWINGS

FIGS. 1A-1D show generation of anti-CS1-huOKT3 dbBiTE and binding to MM.1S and T cells. FIG. 1A: non-reducing SDS gel analysis of individual anti-CS1 and OKT3 antibodies before and after Click chemistry. Gel image is from a single run and not from multiple groupings or cropping. M; protein marker. Note: huOKT3 runs as intact 150 kDa on SEC, but on SDS gels reveals a portion is lacking the disulfide bridge for one of the heavy-light chains. FIG. 1B: schematic representation of Click chemistry at cysteine hinge regions resulting in antibody compound formation. FIGS. 1C-1D: flow analysis of dbBiTE binding to MM.1s and human T cells, respectively, at dilutions shown in FIG. 1D.

FIGS. 2A-2N show dbBiTE-armed activated T cells exhibit potent cytotoxic activity. FIGS. 2A-2C: 6-hour degranulation (CD107a) of dbBiTE-armed, activated T cells following incubation with CS1-expressing or knockout (CS1^{KO}) targets. FIGS. 2D-2F: Intracellular cytokine staining for IFN-γ expression in CS1-dbBiTE armed T cells after co-culture with tumors. In each case, percentage CD4/CD8 T cell populations are shown. FIGS. 2G-2I: Intracellular cytokine staining for TNF-α expression in CS1-dbBiTE armed T cells after co-culture with tumors. In each case, percentage CD4/CD8 T cell populations are shown. FIGS. 2J-2N: Cytokine secretion by dbBiTE-armed, activated T cells in culture supernatants. Representative flow cytometry plots are from single donor (n=3). Data shows the mean±SD; P-values determined by two-way ANOVA and Student t test (two-tailed, unpaired). *P<0.05; **P<0.01; ***P<0.001; ****P<0.0001; ns, not significant.

FIGS. 3A-3G show CS1-dbBiTE-armed resting T cells display potent effector activity. FIGS. 3A-3B: CD107a expression of dbBiTE armed resting T cells cocultured with MM.1S targets. FIG. 3C: Long term tumor lysis by armed T cells against specific targets at increasing effector target (e:t) ratios. FIGS. 3D-3E: IFN-γ positive T cells following 24-hour co-culture with MM.1S and MM.1S-CS1^{KO} target lines. FIGS. 3F-3G: TNF-α positive T cells following 24-hour co-culture with MM.1S and MM.1S-CS1^{KO} target lines. Representative flow plots are from single donor. 1 ug/ml dbBiTE (per 10M cells) was used to generate armed T cells (n=3). Data shows the mean±SD; P-values determined by two-way ANOVA. *P<0.05; **P<0.01; ****P<0.0001.

FIGS. 4A-4D show that CS1-dbBiTE promotes T cell activation and differentiation. Expression of CD69 activation and CD62L memory markers after 6 and 24 hours respectively between dbBiTE-armed (FIGS. 4A-4B) activated and (FIGS. 4C-4D) resting T cells, and subsequent incubation with CS1 positive targets (n=3). Data shows the mean±SD; P-values determined by Student t test (two-tailed, unpaired). *P<0.05; **P<0.01; ***P<0.001; ****P<0.0001; ns, not significant.

FIGS. 5A-5H show that effector memory but not central memory dbBiTE-armed T cells mediate specific effector function. Comparison of dbBiTE mediated (FIG. 5A) degranulation and (FIG. 5B) IFN-γ production between effector memory (T_{EM}) and central memory (T_{CM}) T cells following co-culture with MM.1S. FIGS. 5C-5F: T_{EM} cells exhibit superior proliferative capacity compared to TCM in the presence of dbBiTE alone or in the presence of CS1-expressing MM.1S target cells. Effector memory cells (T_{EM}) from patient samples armed with CS1-dbBiTEs display superior. The generations of cell division are shown. FIG. 5C: CD4+ T_{EM} (CD62L− CD45RO+) dbBITE T cells only; FIG. 5D: CD4+ T_{EM} (CD62L− CD45RO+) dbBITE T cells+MM.1S; FIG. 5E: CD4+ T_{CM} (CD62L+ CD45RO+) dbBITE T cells only; and FIG. 5G: CD4+ T_{CM} (CD62L+ CD45RO+) dbBITE T cells+MM.1S. CD107a expression (FIG. 5G) and IFN-γ positive cells (FIG. 5H), when co-cultured with multiple myeloma target lines MM.1S and U266B. Representative plots are from a single donor (n=3). Data shows the mean±SD; P-values determined by two-way ANOVA. *P<0.05; **P; ns, not significant..

FIGS. 6A-6C show anti-tumor activity of dbBiTE-armed T cells in vivo. FIG. 6A: Tumor progression in mice depicted by bioluminescence imaging (BLI) following engraftment and weekly dbBiTE-T cell infusions. 5 mice per group. Representative mice are presented. FIG. 6B: Comparison of flux curves (Photons/Sec) between untreated and CS1-dbBiTE-T cell treated conditions. FIG. 6C: Residual GFP⁺ tumor cells in bone marrow following tissue harvest from respective conditions shown. MM.1S or MM.1S-CS1KO cells were engrafted by intra-tibial injection on day 0 and tumor progression was monitored weekly for 5 weeks.

FIGS. 7A-7E show the scheme and results for in vivo testing of CS1-dbBiTE. FIG. 7A shows the treatment regimen wherein the concentration of CS1dbBiTE administered to mice was 50 μg/mL. FIGS. 7B-7D provide results showing that the CS1dbBiTE-armed T cells exhibited anti-tumor efficacy in multiple myeloma. FIG. 7E provides results showing that the CS1dbBiTE-armed T cells increased survival in mice.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The term “CD3” refers to any of the recombinant or naturally-occurring forms of the Cluster of Differentiation 3 (CD3) proteins or variants or homologs thereof that comprise the CD3 complex that mediates signal transduction and maintains CD3 complex activity (e.g. within at least 50%, 80%, 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3 proteins in the CD3 complex. In embodiments, the CD3 protein is substantially identical to the protein identified by the UniProt reference number P04234 or a variant or homolog having substantial identity thereto. In embodiments, the CD3 protein is substantially identical to the protein identified by the UniProt reference number P09693 or a variant or homolog having substantial identity thereto. In embodiments, the CD3 protein is substantially identical to the protein identified by the UniProt reference number P07766 or a variant or homolog having substantial identity thereto.

The term “CS-1” is used interchangeably with the terms “CS1,” “CD319,” “CRACC,” and “SLAMF7,” and refers to a homophilic receptor member of the Signaling Lymphocyte Activation Molecule Family (SLAMF7). It is expressed on NK cells, CD8+ T lymphocytes, B lymphocytes, and mature dendritic cells. The human CS1 gene is located on the long arm of chromosome 1 at 1q23-24 between CD48 and CD229. Human NK cells express two splice variants of CS1: CS1-S, which lacks the intracellular domain for activation, and the CS1-L, which contains the intracellular domain for activating NK cytotoxicity. Both isoforms of CS1 are membrane bound forms. However, only the CS1-L isoform is expressed in B cells and signaling through CS1 induces B cell proliferation and autocrine secretion. CS1 is upregulated in MM and it is implicated in the uncontrolled proliferation of MM cells.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect and human cells.

The term “immune cell” as used herein refers to a cell that is part of the immune system and helps the body fight infections and other diseases. Immune cells develop from stem cells in the bone marrow and differentiate into different types of white blood cells. These include granulocytes (neutrophils, eosinophils, and basophils), mast cells, monocytes, macrophages, dendritic cells, natural killer cells, and lymphocytes (B cells and T cells). Basophils, eosinophils and mast cells are important for host defense against parasites. They also are involved in allergic reactions. Neutrophils phagocytose bacteria, and degrade them inside vesicles. Monocytes develop into macrophages and degrade bacteria. Upon activation, monocytes and macrophages coordinate an immune response by notifying other immune cells of the problem. Neutrophils accumulate within minutes at sites of local tissue injury. Dendritic cells are antigen-presenting cells (APCs), and are responsible for processing large molecules into antigens, which, when presented with the appropriate major histocompatibility complex (MHC) on the APCs, are then recognized by adaptive B or T cells. Natural killer (NK) cells are important for recognizing and killing virus-infected cells or tumor cells. They contain intracellular compartments called granules, which are filled with proteins that can form holes in the target cell and also cause their apoptosis. B cells present antigens to T cells, and produce antibodies to neutralize or destroy infectious microbes. T cells are divided into two broad categories: CD8+ T cells or CD4+ T cells, based on which protein is present on the cell's surface. T cells carry out multiple functions, including killing infected cells and activating or recruiting other immune cells. CD8+ T cells also are called cytotoxic T cells or cytotoxic lymphocytes (CTLs). The four major CD4+ T-cell subsets are TH1, TH2, TH17, and Treg, with “TH” referring to “T helper cell.” TH1 cells are critical for coordinating immune responses against intracellular microbes, especially bacteria. TH2 cells are important for coordinating immune responses against extracellular pathogens. TH17 cells are named for their ability to produce interleukin 17 (IL-17), a signaling molecule that activates immune and non-immune cells. TH17 cells are important for recruiting neutrophils. Regulatory T cells (Tregs) monitor and inhibit the activity of other T cells.

The term “genetically-engineered immune cell” as used herein refers to an immune cell having genetically altered DNA. For example, a transfer of genes into the immune cell is used to produce a genetically-engineered immune cell. For example, one base pair modification is used to produce a genetically-engineered immune cell. For example, extracting DNA from another organism's genome and combining it with DNA of that individual is used to produce a genetically-engineered immune cell. The DNA may be either isolated and copied or artificially synthesized. A construct is usually created and used to insert this DNA into the host cell. The construct may include a promoter and terminator region, which initiate and end transcription. The gene may also be modified for better expression or effectiveness. Modifications of the gene may be carried out using recombinant DNA techniques, such as restriction digests, ligations and molecular cloning. A number of techniques known in the art may be used to insert genetic material into the host genome of the immune cell. In embodiments, a genetically-engineered immune cell may be a CAR T cell.

The term “CAR T cell” refers to a T cell that expresses an antigen receptor. CAR T cells may be produced using methods well known in the art. For example, T cells isolated from a patient may be transduced with a nucleic acid encoding a CAR. Following transduction, the cells may be activated. Once activated, cells may be induced to develop into specialized cellular subtypes (e.g. cytotoxic T cells or regulatory T cells) by treating with, for example, specific mixtures of cytokines. CAR T cell populations may be expanded using techniques well known in the art.

The term “OKT3 antibody” refers to a murine monoclonal antibody which recognizes an epitope on the epsilon-subunit within the human CD3 complex. In embodiments, the OKT3 antibody possesses potent immunosuppressive properties in vivo and is effective in the treatment of renal, heart, and liver allograft rejection. The term “humanized OKT3 antibody” refers to a humanized version of an OKT3 antibody. Humanization typically involves CDR grafting of a murine hybridoma. In embodiments, the humanized OKT3 antibody is a humanized version of the gOKT3-5 antibody (Adair et al., 1994 Hum Antibod Hybridomas). In embodiments, the genes encoding the gOKT3-7 variant, composed of the gLC and gHG, are synthesized with human IgG1/k immunoglobulin constant domains, cloned into the pEE12/6 glutamine synthetase vector (Lonza Biologics, Cambridge, UK) and transiently expressed in EXPI293 cells (Thermo Fisher Scientific, Waltham, MA). The secreted antibody can be purified by Protein rA (Millipore Sigma, St Louis, MO) and/or ceramic hydroxyapatite chromatography (Bio-Rad Laboratories, Hercules, CA). In embodiments, the humanized OKT3 antibody stimulates T cell proliferation.

The term “elotuzumab” refers to the humanized immunoglobulin G1 immunostimulatory antibody targeted against CS1. Elotuzumab works by activating natural killer cells, mediating antibody-dependent cell-mediated cytotoxicity (ADCC), and enhancing cytotoxicity by promoting CS1-CS1 interactions between NK cells and CS1+ target cells independent of ADCC. In embodiments, elotuzumab comprises a light chain variable domain which comprises CDR L1 as set forth in SEQ ID NO:15, CDR L2 as set forth in SEQ ID NO:16, and CDR L3 as set forth in SEQ ID NO:17; and a heavy chain variable domain comprising CDR H1 as set forth in SEQ ID NO:19, CDR H2 as set forth in SEQ ID NO:20, and CDR H3 as set forth in SEQ ID NO:21. In embodiments, elotuzumab comprises a light chain variable domain having SEQ ID NO:14 and a heavy chain variable domain having SEQ ID NO:18. In embodiments, elotuzumab comprises the light chain set forth in SEQ ID NO:12 and the heavy chain set forth in SEQ ID NO:13. Elotuzumab is commercially available as EMPLICITI® from Bristol Myers Squibb. The amino acid sequence of elotuzumab is described in U.S. Pat. No. 8,603,477 and https://go.drugbank.com/drugs/DB06317, the disclosures of which are incorporated by reference herein in their entirety.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The term “F(ab)′₂” is used interchangeably with “Fab dimer.” The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). The term “Fab′ monomer” is used interchangeably with “Fab” and “or an antigen-binding fragment.” While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (e.g., McCafferty et al., Nature 348:552-554 (1990)).

Antibodies are large, complex proteins with an intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat et al, Sequences of Proteins of Immunological Interest, U.S. Department of Health and Human Services, 1987. The part of a variable region not contained in the CDRs is called the framework (“FR”), which forms the environment for the CDRs.

An exemplary antibody structural unit comprises a tetramer. Each tetramer is composed of two identical pairs of polypeptide chains, each pair having one “light” and one “heavy” chain. The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable light chain provided herein includes in N-terminal to C-terminal direction a CDR L1, a CDR L2 and a CDR L3.

The term “hinge” or “hinge region” refers to a flexible amino acid stretch between the C_{H}1 and C_{H}2 heavy chains of an antibody, which typically links the two chains by disulfide bonds. The hinge may be rich in cysteine and proline amino acid residues. In embodiments, the disulfide bonds in the hinge may be reduced by a reducing agents, for example tris(2-carboxyethyl)phosphine (TCEP) or dithiothreitol (DTT). In embodiments, the reduced cysteine residues in the hinge may be conjugated to a chemical moiety including a click chemistry reactive functional group. For example, the chemical moiety may be bromoacetamido-dibenzoazacyclooctyne (DBCO) or bromoacetamido-(ethylene glycol).-amido-DBCO. In embodiments, the reduced cysteine is a part of a reactive chemical group side chain.

The term “epitope of an antibody” refers to the region of an antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30%, and preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “dual specific bivalent T-cell engager” is used interchangeably with “dbBITE,” “dbBiTe,” “bispecific antibody compound,” “dual specific bivalent BiTE,” and “antibody compound.”

The term “humanized antibody’ refers to an antibody having one or more amino acid residues introduced into it from a source which is non-human. These non-human amino acid residues are often referred to as import residues, which are typically taken from an import variable domain. Methods for humanizing or primatizing non-human antibodies are well known in the art. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologies. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “fragment” means a portion of an antibody, a polypeptide, or a polynucleotide that is less than the entire antibody, polypeptide, or polynucleotide. A “functional fragment” of a protein, e.g., EGF, CEA, TAG-72, is a fragment of the polypeptide that is shorter than the full-length, immature, or mature polypeptide and has at least 50% (e.g., at least 60%, 70%, 80%, 90%, 95%, 98%, 99%, or even 100% or more) of the activity of full-length mature reference protein. Fragments of interest can be made by recombinant, synthetic, or proteolytic digestive methods.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction. A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The terms “polypeptide,” “peptide,” and “protein” are used interchangeably herein to refer to a polymer of amino acid residues. The polymer of amino acids may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence. The terms “numbered with reference to” or “corresponding to,” when used in the context of the numbering of a given amino acid or nucleotide sequence, refers to the numbering of the residues of a specified reference sequence when the given amino acid or nucleotide sequence is compared to the reference sequence.

An amino acid residue in a protein “corresponds” to a given residue when it occupies the same essential structural position within the protein as the given residue. For example, a selected residue in a selected protein corresponds to specific position (e.g., A100) of a protein when the selected residue occupies the same essential spatial or other structural relationship as that specific position (e.g., A100) of the protein. In embodiments, where a selected protein is aligned for maximum homology with the protein, the position in the aligned selected protein aligning with that specific position (e.g., A100) is said to correspond to that specific residue (e.g., A100). Instead of a primary sequence alignment, a three dimensional structural alignment can also be used, e.g., where the structure of the selected protein is aligned for maximum correspondence with the protein and the overall structures compared. In this case, an amino acid that occupies the same essential position as that specific position (e.g., A100) in the structural model is said to correspond to the that specific position residue (e.g., A100).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, or at least 65%, 70%, 75%, 80%, 85%, 90%, 91%, 92%, 93%, 94%, 95%, 96%, 97%, 98%, 99% identity over a specified region, when compared and aligned for maximum correspondence over a comparison window or designated region) as measured using a BLAST or BLAST 2.0 sequence comparison algorithms with default parameters described below, or by manual alignment and visual inspection (e.g., NCBI web site ncbi.nlm.nih.gov/BLAST/ or the like). Such sequences are then said to be “substantially identical.” The definition also includes sequences that have deletions and/or additions, as well as those that have substitutions. Algorithms can account for gaps and the like. Preferably, identity exists over a region that is at least about 25 amino acids or nucleotides in length, or more preferably over a region that is 50-100 amino acids in length.

“Click chemistry” is used in accordance with its well understood meaning in chemistry and refers generally to reactions that are high yielding, wide in scope, create only by-products that can be removed without chromatography, are stereospecific, simple to perform, and can be conducted in easily removable or benign solvents. A click chemistry reactive functional group is a chemical group capable of reacting (e.g., covalently) with a complementary click chemistry reactive functional group in a “click chemistry” reaction. Several examples of reactions have been identified that fulfill these criteria, thermodynamically-favored reactions that lead specifically to one product, such as for example, nucleophilic ring opening reactions of epoxides and aziridines, non-aldol type carbonyl reactions, such as formation of hydrazones and heterocycles, additions to carbon-carbon multiple bonds, such as oxidative formation of epoxides and Michael additions, and cycloaddition reactions. Chemical synthesis of compositions by joining small modular units using conjugate (“click”) chemistry is well known in the art.

The term “reactive functional group” or a “click chemistry reactive functional group” as used herein refers to a chemical moiety (e.g., a first reactive functional group), which is capable of interacting (e.g., forming a bond with, covalently or non-covalently) with a second, optionally different, chemical moiety (e.g., a second reactive functional group, a complementary reactive functional group). In embodiments, the reactive functional group is a chemical moiety capable of reacting with a second reactive (e.g. a complementary reactive functional group) to form a covalent attachment (e.g. a linker or bond). In embodiments, the reactive functional group is a bioconjugate reactive group capable of interacting (e.g., covalently) with a complementary bioconjugate reactive group (e.g., complementary reactive functional group). In embodiments, a reactive functional group is a click chemistry reactive functional group. In embodiments, the reactive functional group is capable of covalently interacting with a second chemical moiety (e.g., complementary reactive functional group). In embodiments, the reactive functional group is capable of non-covalently interacting with a second chemical moiety (e.g., complementary reactive functional group). Non-limiting examples of a reactive functional group include biotin, azide, trans-cyclooctene (TCO). In embodiments, a reactive functional group (e.g., biotin moiety) interacts non-covalently with a complementary reactive functional group (e.g., streptavidin moiety). In embodiments, a reactive functional group (e.g., azide moiety, trans-cyclooctene (TCO) moiety, phenyl boric acid (PBA) moiety) covalently binds a complementary reactive functional group (e.g., dibenzocyclooctyne (DBCO) moiety, tetrazine (TZ) moiety, salicylhydroxamic acid (SHA) moiety).

The term “reactive chemical group side chain” as used herein refers to a chemical moiety (e.g., a first reactive chemical group side chain) covalently bonded (or bound) to an amino acid alpha carbon atom (e.g., an alpha carbon atom of a peptide residue or protein residue) and including a reactive functional group. In embodiments, the reactive functional group is covalently bonded to the remnant of a natural amino acid side chain resulting from the reaction of a natural amino acid side chain with a compound having the reactive functional group. In embodiments, the reactive chemical group side chain includes a reactive functional group wherein the reactive functional group is a bioconjugate reactive group capable of interacting (e.g., covalently) with a complementary bioconjugate reactive group (e.g., complementary reactive chemical group side chain). In embodiments, the reactive functional group within the reactive chemical group side chain is a click chemistry reactive functional group. In embodiments, the reactive functional group within the reactive chemical group side chain is a bioconjugate reactive group capable of interacting (e.g., covalently) with a complementary bioconjugate reactive group (e.g., complementary reactive chemical group side chain). In embodiments, the reactive functional group within the reactive chemical group side chain includes a click chemistry reactive functional group. In embodiments, the reactive functional group within the reactive chemical group side chain is capable of covalently bonding with a second chemical moiety (e.g., complementary reactive chemical group side chain).

The term “bioconjugate reactive moiety” and “bioconjugate reactive group” refers to a moiety or group capable of forming a bioconjugate (e.g., covalent linker or bond) as a result of the association between atoms or molecules of two bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are known in the art.

Useful reactive moieties or functional groups (e.g. used for bioconjugate chemistries including “click chemistries” as known in the art) herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g. phosphines) to form, for example, phosphate diester bonds; (n) azides coupled to alkynes using copper catalyzed cycloaddition click chemistry or other click chemistry complementary reactive groups; (o) biotin conjugate can react with avidin or strepavidin to form a avidin-biotin complex or streptavidin-biotin complex; and (p) sulfones, for example, vinyl sulfone.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Exemplarf unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable non-cyclic straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —(CH₂)₂O—CH₃, —(CH₂)₂—NH—CH₃, —(CH₂)₃—OH, —CH₂—NH₂, —CH₂—NO₂, —(CH₂)₂—N(CH₃)—CH₃, —CH₂—S—CH₂—CH₃, —S(O)—CH₃, —(CH₂)₂—S(O)₂—CH₃, —CH═CHO—CH₃, —Si(CH₃)₃, —CH₂—CH═N—OCH₃, —O—CH₃, —CH═CH—N(CH₃)—CH₃, —O—CH₂—CH₃, and —CN. Up to two or three heteroatoms may be consecutive, such as, for example, —CH₂—NH—OCH₃ and —CH₂—O—Si(CH₃)₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂—, —O—CH₂—CH₂—NH—CH₂—, —O—(CH₂)₃—O—PO₃—, —O—(CH₂)—O—PO₃—, —O—(CH₂)₂—O—PO₃—, —O—(CH₂)₄—O—PO₃—, and the like. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted as excluding specific heteroalkyl groups, such as —NR′R″ or the like.

The terms “cycloalkyl” and “heterocycloalkyl,” by themselves or in combination with other terms, mean, unless otherwise stated, cyclic non-aromatic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently (e.g., biphenyl). A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “oxo” means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″″, —S(O)R′, —S(O)₂R′, —S(O)₂NR′R″, —NRSO₂R′, —NR′NR″R′″″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In embodiments, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from: (i) oxo, halogen, —CF₃, —CN, —OH, —NH₂, —COOH, —NO₂, —CONH₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, heteroaryl, substituted with at least one substituent selected from: (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl.

In embodiments, each substituted group described in the compounds herein is substituted with at least one substituent group. More specifically, in embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In aspects of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In aspects of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

“Treating” or “treatment” as used herein refers to any approach for obtaining beneficial clinical results in a patient's condition. Beneficial clinical results can include, but are not limited to, alleviation or amelioration of one or more symptoms or conditions, diminishment of the extent of a disease, stabilizing (i.e., not worsening) the state of disease, delay or slowing of disease progression, amelioration or palliation of the disease state, and remission, whether partial or total and whether detectable or undetectable. In other words, “treatment” as used herein includes any cure or amelioration of a disease. Treatment may inhibit the disease's spread; relieve the disease's symptoms; fully or partially remove the disease's underlying cause; shorten a disease's duration; or do a combination of these things. Treatment methods include administering to a patient a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In instances, chronic administration may be required. For example, the compositions are administered to the patient in an amount and for a duration sufficient to treat the patient. In embodiments, “treating” does not include “preventing.”

“Patient” or “patient in need thereof” refers to a living organism suffering from or prone to a disease or condition that can be treated by administration of an antibody compound, antibody, compound, or pharmaceutical composition as provided herein. Non-limiting examples include humans, other mammals (e.g., bovine, rats, mice, dogs, monkeys, cats), and non-mammalian animals. In embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The term “therapeutically effective amount” refers to that amount of the therapeutic agent sufficient to treat the disorder, as described above.

The term “administering” is used in accordance with its plain and ordinary meaning and includes oral administration, administration by inhalation, administration by suppository, topical contact, intravenous, parenteral, intraperitoneal, intramuscular, intralesional, intrathecal, intranasal or subcutaneous administration, or the implantation of a slow-release device, e.g., a mini-osmotic pump, to a patient. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Contacting” is used in accordance with its plain ordinary meaning and refers to the process of allowing at least two distinct species (e.g. chemical compounds including antibodies and/or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

The terms “isolate” or “isolated” when applied to a cell, nucleic acid, virus, or protein, denotes that the cell, nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. An immune cell that is the predominant species present in a preparation is substantially purified.

A “detectable agent” or “detectable moiety” is a compound or composition detectable by appropriate means such as spectroscopic, photochemical, biochemical, immunochemical, chemical, magnetic resonance imaging, or other physical means. A detectable moiety is a monovalent detectable agent or a detectable agent bound (e.g. covalently and directly or via a linking group) with another compound, e.g., a nucleic acid.

“Biological sample” or “sample” refer to materials obtained from or derived from a patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, immune cells, hematopoietic cells, fibroblasts, macrophages, T cells, etc.

The term “disease” or “condition” refers to a state of being or health status of a patient that is being treated with the compounds or methods provided herein. In embodiments, the disease is cancer, an autoimmune disease, an inflammatory disease, or an infectious disease. In embodiments, cancer refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma. In embodiments, the disease is cancer. In embodiments, the disease is multiple myeloma.

The term “cancer” is used in accordance with its plain ordinary meaning and refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “multiple myeloma” refers to a cancer of plasma cells. Normal plasma cells are found in the bone marrow and are part of the immune system. The immune system is made up of several types of cells and includes lymphocytes (lymph cells), which comprise T cells and B cells. Lymphocytes are in many areas of the body, such as lymph nodes, the bone marrow, the intestines, and the bloodstream. When B cells respond to an infection, they mature and change into plasma cells and produce antibodies. Cancerous plasma cells make an abnormal antibody known as monoclonal immunoglobulin, monoclonal protein (M-protein), M-spike, or paraprotein. The overgrowth of plasma cells in the bone marrow in multiple myeloma leads to low blood count, causing anemia (a shortage of red blood cells), thrombocytopenia (low level of platelets), and leukopenia (a shortage of normal white blood cells). The buildup of M protein in the blood and urine can damage the kidneys and other organs. Bone damage can cause bone pain and osteolytic lesions, with consequent increased risk of fractures, and can also lead to a serious condition called hypercalcemia (increased levels of calcium in the blood). Monoclonal gammopathy of undetermined significance (MGUS) is a plasma cell neoplasm diagnosed when a small amount of M protein is detected in the blood, but no other criteria for a solitary plasmacytoma or multiple myeloma diagnosis (such as a tumor, multiple lesions or symptoms) are present. MGUS occurs in about 1% of the general population. Solitary (or isolated) plasmacytoma occurs when a single group of malignant myeloma cells grows inside or outside of bone. An “extramedullary plasmacytoma” grows outside the bone. An “isolated plasmacytoma of the bone” grows within the bone. Smoldering multiple myeloma (SMM) is a precancerous form of multiple myeloma that typically accounts for about 15% of newly diagnosed multiple myeloma cases. It is diagnosed when low levels of M protein are found in the blood and a slightly increased number of plasma cells are found in the bone marrow. Management of multiple myeloma includes immediate treatment with myeloma drugs, bisphosphonates for patients with bone loss and options for clinical trials. The main types of drug therapies used to treat multiple myeloma are proteasome inhibitors, immunomodulatory drugs (IMiDs), steroids, histone deacetylase (HDAC) inhibitors, antibodies, radiation therapy and chemotherapy.

The singular forms “a,” “an,” and “the” include plural referents unless the context clearly dictates otherwise.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

### Antibody Compounds

Provided herein are antibody compounds comprising an anti-immune cell antibody covalently bound to an anticancer antibody. In embodiments, the antibody compounds comprise an anti-immune cell antibody covalently bound to a CS-1-binding antibody. In embodiments, the antibody compounds comprise elotuzumab covalently bound to an OKT3 antibody. In embodiments, the antibody compounds comprise a cysteine residue in elotuzumab covalently bound to a cysteine residue in an OKT3 antibody. In embodiments, the antibody compounds comprise a cysteine in the hinge region of elotuzumab covalently bound to a cysteine in the hinge region of an OKT3 antibody. In embodiments, the antibody compounds comprise elotuzumab covalently bound to a humanized OKT3 antibody. In embodiments, the antibody compounds comprise a cysteine residue in elotuzumab covalently bound to a cysteine residue in a humanized OKT3 antibody. In embodiments, the antibody compounds comprise a cysteine residue in the hinge region of elotuzumab covalently bound to a cysteine residue in the hinge region of a humanized OKT3 antibody.

In embodiments, the antibody compounds comprise an elotuzumab F(ab′)₂ fragment covalently bound to an OKT3 antibody F(ab′)₂ fragment. In embodiments, the antibody compounds comprise a cysteine residue of an elotuzumab F(ab′)₂ fragment covalently bound to a cysteine residue of an OKT3 antibody F(ab′)₂ fragment. In embodiments, the cysteine residue is in the hinge region. In embodiments, the antibody compounds comprise the cysteine hinge region of an elotuzumab F(ab′)₂ fragment covalently bound to the cysteine hinge region of an OKT3 antibody F(ab′)₂ fragment. In embodiments, the antibody compounds comprise an elotuzumab F(ab′) fragment covalently bound to an OKT3 antibody F(ab′) fragment. In embodiments, the antibody compounds comprise a cysteine residue of an elotuzumab F(ab′) fragment covalently bound to a cysteine residue of an OKT3 antibody F(ab′) fragment. In embodiments, the cysteine residue is in the hinge region. In embodiments, the antibody compounds comprise the cysteine hinge region of an elotuzumab F(ab′) fragment covalently bound to the cysteine hinge region of an OKT3 antibody F(ab′) fragment.

In embodiments, the antibody compounds comprise an elotuzumab F(ab′)₂ fragment covalently bound to a humanized OKT3 antibody F(ab′)₂ fragment. In embodiments, the antibody compounds comprise a cysteine residue of an elotuzumab F(ab′)₂ fragment covalently bound to a cysteine residue of a humanized OKT3 antibody F(ab′)₂ fragment. In embodiments, the cysteine residue is in the hinge region. In embodiments, the antibody compounds comprise the cysteine hinge region of an elotuzumab F(ab′)₂ fragment covalently bound to the cysteine hinge region of a humanized OKT3 antibody F(ab′)₂ fragment. In embodiments, the antibody compounds comprise an elotuzumab F(ab′) fragment covalently bound to a humanized OKT3 antibody F(ab′) fragment. In embodiments, the antibody compounds comprise a cysteine residue of an elotuzumab F(ab′) fragment covalently bound to a cysteine residue of a humanized OKT3 antibody F(ab′) fragment. In embodiments, the cysteine residue is in the hinge region. In embodiments, the antibody compounds comprise the cysteine hinge region of an elotuzumab F(ab′) fragment covalently bound to the cysteine hinge region of a humanized OKT3 antibody F(ab′) fragment.

In embodiments, the compounds provided herein include two antibodies (i.e., two intact antibodies), each of which retains both Fab′ domains. In embodiments, the compounds include two antibodies each of which retains bivalent binding ability to antigens. In embodiment, each antibody comprises a hinge region. In embodiments, the hinge region of each antibody is a cysteine hinge region. In embodiments, the hinge region of each antibody is reduced prior to formation of the antibody compound. In embodiments, the CS-1-binding antibody and the anti-immune cell antibody are connected through their hinge regions. In embodiments, click chemistry is used to covalently bind the CS-1-binding antibody and the anti-immune cell antibody.

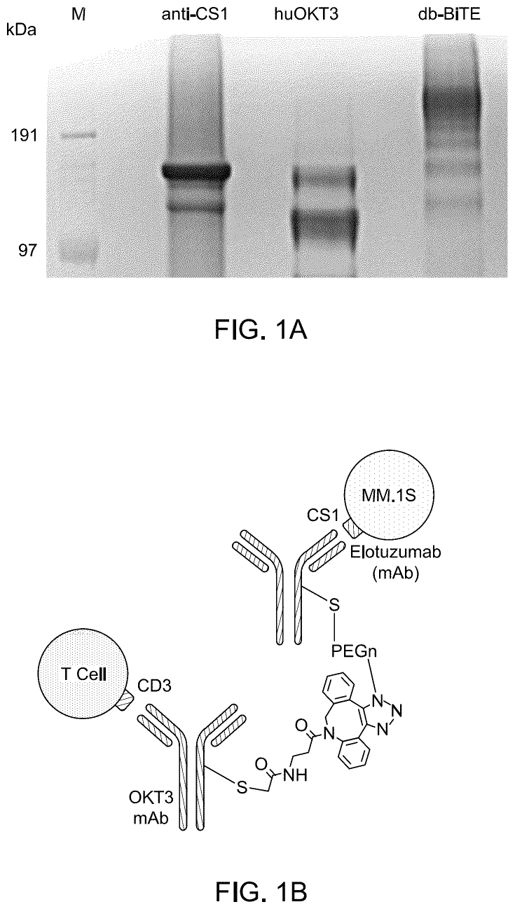

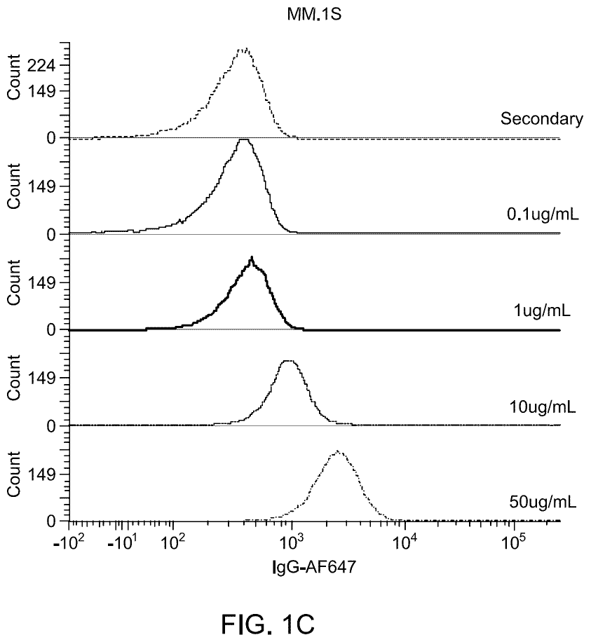

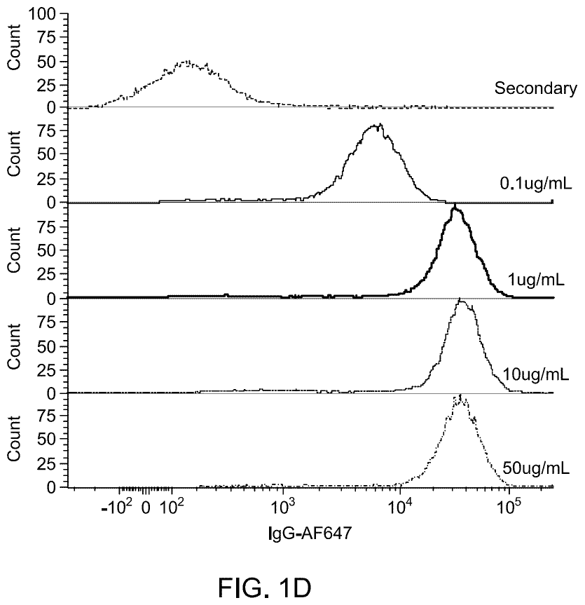

In embodiments, the compounds provided herein include two antibodies, i.e., elotuzumab and a humanized OKT3 antibody (FIG. 1B). In embodiments, the compounds comprise two F(ab′)₂ fragments, i.e., an elotuzumab F(ab′)₂ fragment and a humanized OKT3 antibody F(ab′)₂ fragment. In embodiments, the compounds comprise two F(ab′) fragments, i.e., an elotuzumab F(ab′) fragment and a humanized OKT3 antibody F(ab′) fragment. In embodiments, the compounds comprise: (i) an elotuzumab antibody, an elotuzumab F(ab′)₂ fragment, or an elotuzumab F(ab′) fragment, and (ii) a humanized OKT3 antibody, a humanized OKT3 antibody F(ab′)₂ fragment, or a humanized OKT3 antibody F(ab′) fragment; wherein (i) is covalently bonded to (ii). In embodiments, the compounds comprise: (i) an elotuzumab antibody, an elotuzumab F(ab′)₂ fragment, or an elotuzumab F(ab′) fragment, and (ii) a humanized OKT3 antibody, a humanized OKT3 antibody F(ab′)₂ fragment, or a humanized OKT3 antibody F(ab′) fragment; wherein the cysteine hinge region of (i) is covalently bonded to the cysteine hinge region of (ii).

In embodiments, the CS-1-binding antibody and the anti-immune cell antibody are alkylated at their sulfhydryl reduced hinge regions. In embodiments, two complimentary click reagents are used to form the compounds provided herein. In embodiments, the complimentary click reagents comprise alkyne reagents and azide-bearing compounds. In embodiments, the alkyne reagents comprise alkynes bearing dibenzocyclooctyne (DBCO) or bicyclo[6.1.0]nonyne (BCO). In embodiments, the alkyne reagent is an alkyne bearing DBCO. In embodiments, the alkyne reagent is an alkyne bearing BCO. In embodiments, the azide-bearing compounds comprise organic azides and PEG azides. In embodiments, the azide-bearing compounds are organic azides. In embodiments, the azide-bearing compounds are PEG azides. In embodiments, the PEG azide is bromoacetamido-PEG₅-azide.

In embodiments, a cysteine residue in the anti-CD3 antibody is covalently bound to a cysteine residue in the CS-1-binding antibody via a bond, an amino acid linking group, or a chemical linking group. In embodiments, the chemical linking group has the formula -L¹-L²-L³- wherein L¹ is linked to a sulfur group in the CD3-binding antibody and L³ is linked to a sulfur group in the CS-1-binding antibody.

In embodiments, a cysteine residue in the hinge region of the anti-CD3 antibody is covalently bound to a cysteine residue in the hinge region of the CS-1-binding antibody via a bond, an amino acid linking group, or a chemical linking group. In embodiments, the chemical linking group has the formula -L¹-L²-L³- wherein L¹ is linked to a sulfur group in the hinge region of the CD3-binding antibody and L³ is linked to a sulfur group in the hinge region of the CS-1-binding antibody.

In embodiments, -L¹-L²-L³- are each independently a bond, a nucleic acid, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a combination of two or more thereof.

In embodiments, L³ is unsubstituted heteroalkylene. In embodiments, L³ is polyethylene glycol. In embodiments, L³ is polyethylene glycol having the structure —W—(OCH₂CH₂)ₙ, where W is a bond or unsubstituted C₁₋₆ akylene, and n is an integer from 1 to about 200. In embodiments, W is unsubstituted C₁₋₄ akylene. In embodiments, W is unsubstituted C₁.3 akylene. In embodiments, W is unsubstituted C₁₋₂ akylene. In embodiments, W is —CH₂—. In embodiments, W is —(CH₂)₂—. In embodiments, W is —(CH₂)₃—. In embodiments, W is —(CH₂)₄—. In embodiments, W is —(CH₂)₅—. In embodiments, W is —(CH₂)₆—. In embodiments, W is a bond. In embodiments, L³ is —CH₂CH₂—[—OCH₂CH₂—]ₙ—. In embodiments, n is 1 to about 100. In embodiments, n is 1 to about 60. In embodiments, n is 1 to about 55. In embodiments, n is 1 to about 50. In embodiments, n is 1 to about 45. In embodiments, n is 1 to about 40. In embodiments, n is 1 to about 35. In embodiments, n is 1 to about 30. In embodiments, n is 1 to about 25. In embodiments, n is 1 to about 20. In embodiments, n is 1 to about 15. In embodiments, n is 1 to about 10. In embodiments, n is 1 to about 5. In embodiments, n is about 5 to about 10. In embodiments, n is about 5 to about 15. In embodiments, n is about 5 to about 20. In embodiments, n is about 5 to about 25. In embodiments, n is about 5 to about 35. In embodiments, n is about 5 to about 40. In embodiments, n is about 10 to about 60. In embodiments, n is about 10 to about 50. In embodiments, n is about 10 to about 40, In embodiments, n is about 10 to about 30. In embodiments, n is about 10 to about 20.

In embodiments, L² is a substituted or unsubstituted heteroarylene. In embodiments, L² is a substituted or unsubstituted fused heteroarylene. In embodiments, L² is a substituted or unsubstituted fused heteroarylene having from 2 to 6 rings, wherein each ring is 5 to 8 membered ring. In embodiments, L² is a substituted or unsubstituted fused heteroarylene having from 2 to 4 rings, wherein each ring is 5 to 8 membered ring. In embodiments, L² is unsubstituted fused heteroarylene having from 2 to 5 rings, wherein each ring is 5 to 8 membered ring. In embodiments, L² is

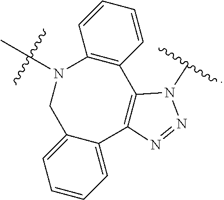 [SMILES: *N1Cc2ccccc2-c2nnn(*)c2-c2ccccc21]

In embodiments, L¹ is substituted heteroalkylene. In embodiments, L¹ is 2 to 12 membered substituted heteroalkylene. In embodiments, L¹ is 2 to 12 membered substituted heteroalkylene comprising one or two nitrogen atom and substituted with 1-3 oxo (═O) groups. In embodiments, L¹ is —CH₂C(═O)NHCH₂CH₂C(═O)—

In embodiments, -L¹-L²-L³- is

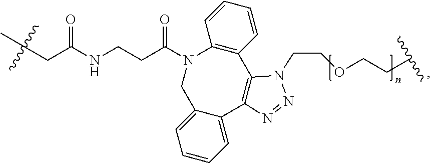

where n is an integer from 1 to 100. In embodiments, n is 1 to 60. In embodiments, n is 1 to 50. In embodiments, n is 1 to 45. In embodiments, n is 1 to 40. In embodiments, n is 1 to 35. In embodiments, n is 1 to 30. In embodiments, n is 1 to 25. In embodiments, n is 1 to 20. In embodiments, n is 1 to 15. In embodiments, n is 1 to 10. In embodiments, n is 1 to 5.

In embodiments, the CS-1-binding antibody comprises a sequence which is at least 80%, at least 85%, at least 90%, at least 95%, at least 98%, or 100% identical to the sequence of elotuzumab. In embodiments, the CS-1-binding antibody comprises a sequence which is at least 80% identical to the sequence of elotuzumab. In embodiments, the CS-1-binding antibody comprises a sequence which is at least 85% identical to the sequence of elotuzumab. In embodiments, the CS-1-binding antibody comprises a sequence which is at least 90% identical to the sequence of elotuzumab. In embodiments, the CS-1-binding antibody comprises a sequence which is at least 95% identical to the sequence of elotuzumab. In embodiments, the CS-1-binding antibody comprises a sequence which is at least 98% identical to the sequence of elotuzumab. In embodiments, the CS-1-binding antibody comprises a sequence which is 100% identical to the sequence of elotuzumab. In embodiments, the CS-1 binding antibody binds to the same epitope as elotuzumab.

In embodiments, the CS-1-binding antibody comprises: (i) a light chain variable domain CDR L1 as set forth in SEQ ID NO:15, CDR L2 as set forth in SEQ ID NO:16, and CDR L3 as set forth in SEQ ID NO:17; and (ii) a heavy chain variable domain CDR H1 as set forth in SEQ ID NO:19, CDR H2 as set forth in SEQ ID NO:20, and CDR H3 as set forth in SEQ ID NO:21.

In embodiments, the CS-1-binding antibody comprises: (i) a light chain variable domain CDR L1 having a sequence that is at least 95% identical, at least 96% identical, at least 97% identical, at least 98% identical, or at least 99% identical to SEQ ID NO:15, CDR L2 having a sequence that is at least 95% identical, at least 96% identical, at least 97% identical, at least 98% identical, or at least 99% identical to SEQ ID NO:16, and CDR L3 having a sequence that is at least 95% identical, at least 96% identical, at least 97% identical, at least 98% identical, or at least 99% identical to SEQ ID NO:17; and (ii) a heavy chain variable domain CDR H1 having a sequence that is at least 95% identical, at least 96% identical, at least 97% identical, at least 98% identical, or at least 99% identical to SEQ ID NO:19, CDR H2 having a sequence that is at least 95% identical, at least 96% identical, at least 97% identical, at least 98% identical, or at least 99% identical to SEQ ID NO:20, and CDR H3 having a sequence that is at least 95% identical, at least 96% identical, at least 97% identical, at least 98% identical, or at least 99% identical to SEQ ID NO:21.

In embodiments, the CS-1-binding antibody comprises: (i) a light chain variable domain having SEQ ID NO:14; and (ii) a heavy chain variable domain having SEQ ID NO:18.

In embodiments, the CS-1-binding antibody comprises: (i) a light chain variable domain having at least 90% sequence identity to SEQ ID NO:14; and (ii) a heavy chain variable domain having at least 90% sequence identity to SEQ ID NO:18. In embodiments, the CS-1-binding antibody comprises: (i) a light chain variable domain having at least 95% sequence identity to SEQ ID NO:14; and (ii) a heavy chain variable domain having at least 95% sequence identity to SEQ ID NO:18. In embodiments, the CS-1-binding antibody comprises: (i) a light chain variable domain having at least 96% sequence identity to SEQ ID NO:14; and (ii) a heavy chain variable domain having at least 96% sequence identity to SEQ ID NO:18. In embodiments, the CS-1-binding antibody comprises: (i) a light chain variable domain having at least 97% sequence identity to SEQ ID NO:14; and (ii) a heavy chain variable domain having at least 97% sequence identity to SEQ ID NO:18. In embodiments, the CS-1-binding antibody comprises: (i) a light chain variable domain having at least 98% sequence identity to SEQ ID NO:14; and (ii) a heavy chain variable domain having at least 98% sequence identity to SEQ ID NO:18. In embodiments, the CS-1-binding antibody comprises: (i) a light chain variable domain having at least 99% sequence identity to SEQ ID NO:14; and (ii) a heavy chain variable domain having at least 99% sequence identity to SEQ ID NO:18. In embodiments where there is less than 100% sequence identity to the light chain variable domain or the heavy chain variable domain, the sequence has 100% identity with the CDRs within the light chain variable domain and the sequence has 100% identity with the CDRs within the heavy chain variable domain.

In embodiments, the CS-1-binding antibody comprises: (i) a light chain having SEQ ID NO:12; and (ii) a heavy chain having SEQ ID NO:13.

In embodiments, the CS-1-binding antibody comprises: (i) a light chain having at least 90% sequence identity to SEQ ID NO:12; and (ii) a heavy chain having at least 90% sequence identity to SEQ ID NO:13. In embodiments, the CS-1-binding antibody comprises: (i) a light chain having at least 95% sequence identity to SEQ ID NO:12; and (ii) a heavy chain having at least 95% sequence identity to SEQ ID NO:13. In embodiments, the CS-1-binding antibody comprises: (i) a light chain having at least 96% sequence identity to SEQ ID NO:12; and (ii) a heavy chain having at least 96% sequence identity to SEQ ID NO:13. In embodiments, the CS-1-binding antibody comprises: (i) a light chain having at least 97% sequence identity to SEQ ID NO:12; and (ii) a heavy chain having at least 97% sequence identity to SEQ ID NO:13. In embodiments, the CS-1-binding antibody comprises: (i) a light chain having at least 98% sequence identity to SEQ ID NO:12; and (ii) a heavy chain having at least 98% sequence identity to SEQ ID NO:13. In embodiments, the CS-1-binding antibody comprises: (i) a light chain having at least 99% sequence identity to SEQ ID NO:12; and (ii) a heavy chain having at least 99% sequence identity to SEQ ID NO:13. In embodiments where there is less than 100% sequence identity to the light chain or the heavy chain, the light chain sequence has 100% sequence identity with the CDRs within the light chain variable domain and the heavy chain sequence has 100% sequence identity with the CDRs within the heavy chain variable domain.

In embodiments, the anti-cancer antibody comprises a CS-1-binding antibody. In embodiments, the CS-1-binding antibody comprises a sequence which is at least 80%, at least 85%, at least 90%, at least 95%, at least 98%, or 100% identical to SEQ ID NO:11. In embodiments, the CS-1-binding antibody comprises a sequence which is at least 80% identical to SEQ ID NO:11. In embodiments, the CS-1-binding antibody comprises a sequence which is at least 85% identical to SEQ ID NO:11. In embodiments, the CS-1-binding antibody comprises a sequence which is at least 90% identical to SEQ ID NO:11. In embodiments, the CS-1-binding antibody comprises a sequence which is at least 95% identical to SEQ ID NO:11. In embodiments, the CS-1-binding antibody comprises a sequence which is at least 98% identical to SEQ ID NO:11. In embodiments, the CS-1-binding antibody comprises SEQ ID NO:11. In embodiments, the CS-1-binding antibody has SEQ ID NO:11.

In embodiments, the anti-immune cell antibody is a T cell antigen-binding antibody. In embodiments, the T cell antigen-binding antibody is an anti-CD3 antibody. In embodiments, the anti-CD3 antibody is an OKT3 antibody. In embodiments, the OKT3 antibody is a humanized OKT3 antibody.

In embodiments, OKT3 antibody comprises: (i) a light chain variable domain comprising CDR L1 as set forth in SEQ ID NO:1, CDR L2 as set forth in SEQ ID NO:2 and CDR L3 as set forth in SEQ ID NO:3; and (ii) a heavy chain variable domain comprising CDR H1 as set forth in SEQ ID NO:4, CDR H2 as set forth in SEQ ID NO:5 and CDR H3 as set forth in SEQ ID NO:6

In embodiments, the humanized OKT3 antibody comprises a light chain variable domain comprising a CDR L1 which is at least 95% identical, at least 96% identical, at least 97% identical, at least 98% identical, or at least 99% identical to SEQ ID NO:1, a CDR L2 which is at least 95% identical, at least 96% identical, at least 97% identical, at least 98% identical, or at least 99% identical to SEQ ID NO:2 and a CDR L3 which is at least 95% identical, at least 96% identical, at least 97% identical, at least 98% identical, or at least 99% identical to SEQ ID NO:3; and a heavy chain variable domain comprising a CDR H1 which is at least 95% identical, at least 96% identical, at least 97% identical, at least 98% identical, or at least 99% identical to SEQ ID NO:4, a CDR H2 which is at least 95% identical, at least 96% identical, at least 97% identical, at least 98% identical, or at least 99% identical to SEQ ID NO:5, and a CDR H3 which is at least 95% identical, at least 96% identical, at least 97% identical, at least 98% identical, or at least 99% identical to SEQ ID NO:6.

In embodiments, the humanized OKT3 antibody comprises: (i) a light chain variable domain having SEQ ID NO:9; and (ii) a heavy chain variable domain having SEQ ID NO:10.

In embodiments, the humanized OKT3 antibody comprises: (i) a light chain variable domain having at least 90% sequence identity to SEQ ID NO:9; and (ii) a heavy chain variable domain having at least 90% sequence identity to SEQ ID NO:10. In embodiments, the humanized OKT3 antibody comprises: (i) a light chain variable domain having at least 95% sequence identity to SEQ ID NO:9; and (ii) a heavy chain variable domain having at least 95% sequence identity to SEQ ID NO:10. In embodiments, the humanized OKT3 antibody comprises: (i) a light chain variable domain having at least 96% sequence identity to SEQ ID NO:9; and (ii) a heavy chain variable domain having at least 96% sequence identity to SEQ ID NO:10. In embodiments, the humanized OKT3 antibody comprises: (i) a light chain variable domain having at least 97% sequence identity to SEQ ID NO:9; and (ii) a heavy chain variable domain having at least 97% sequence identity to SEQ ID NO:10. In embodiments, the humanized OKT3 antibody comprises: (i) a light chain variable domain having at least 98% sequence identity to SEQ ID NO:9; and (ii) a heavy chain variable domain having at least 98% sequence identity to SEQ ID NO:10. In embodiments, the humanized OKT3 antibody comprises: (i) a light chain variable domain having at least 99% sequence identity to SEQ ID NO:9; and (ii) a heavy chain variable domain having at least 99% sequence identity to SEQ ID NO:10. In embodiments where there is less than 100% sequence identity to the light chain variable domain or the heavy chain variable domain, the sequence has 100% identity with the CDRs within the light chain variable domain and the sequence has 100% identity with the CDRs within the heavy chain variable domain.

In embodiments, the humanized OKT3 antibody comprises: (i) a signal sequence as set forth in SEQ ID NO:7 in the light chain variable domain; and (ii) a signal sequence as set forth in SEQ ID NO:8 in the heavy chain variable domain. In embodiments, the signal sequence of SEQ ID NO:7 is at the 5′ end of the light chain variable domain, i.e., set forth as SEQ ID NO:22. In embodiments, the signal sequence of SEQ ID NO:8 is at the 5′ end of the heavy chain variable domain, i.e., set forth as SEQ ID NO:23.

In embodiments, the humanized OKT3 antibody comprises: (i) a signal sequence having at least 90% sequence identity with SEQ ID NO:7 in the light chain variable domain; and (ii) a signal sequence having at least 90% sequence identity with SEQ ID NO:8 in the heavy chain variable domain. In embodiments, the humanized OKT3 antibody comprises: (i) a signal sequence having at least 95% sequence identity with SEQ ID NO:7 in the light chain variable domain; and (ii) a signal sequence having at least 95% sequence identity with SEQ ID NO:8 in the heavy chain variable domain. In embodiments, the humanized OKT3 antibody comprises: (i) a signal sequence having at least 96% sequence identity with SEQ ID NO:7 in the light chain variable domain; and (ii) a signal sequence having at least 96% sequence identity with SEQ ID NO:8 in the heavy chain variable domain. In embodiments, the humanized OKT3 antibody comprises: (i) a signal sequence having at least 97% sequence identity with SEQ ID NO:7 in the light chain variable domain; and (ii) a signal sequence having at least 97% sequence identity with SEQ ID NO:8 in the heavy chain variable domain. In embodiments, the humanized OKT3 antibody comprises: (i) a signal sequence having at least 98% sequence identity with SEQ ID NO:7 in the light chain variable domain; and (ii) a signal sequence having at least 98% sequence identity with SEQ ID NO:8 in the heavy chain variable domain. In embodiments, the humanized OKT3 antibody comprises: (i) a signal sequence having at least 99% sequence identity with SEQ ID NO:7 in the light chain variable domain; and (ii) a signal sequence having at least 99% sequence identity with SEQ ID NO:8 in the heavy chain variable domain. In embodiments, the signal sequence of SEQ ID NO:7 is at the 5′ end of the light chain variable domain, i.e., set forth as SEQ ID NO:22, and the signal sequence of SEQ ID NO:8 is at the 5′ end of the heavy chain variable domain, i.e., set forth as SEQ ID NO:23).

Provided herein are compositions comprising an antibody compound described herein (including embodiments thereof) and an immune cell. In embodiments, the compositions comprise an antibody compound described herein (including embodiments thereof) and a T cell, a B cell, a natural killer cell, a monocyte, a neutrophil, a macrophage, a genetically-engineered immune cell, or a combination of two or more thereof. In embodiments, the compositions comprise an antibody compound described herein (including embodiments thereof) and a T cell, a natural killer cell, a monocyte, a neutrophil, a macrophage, a genetically-engineered immune cell, or a combination of two or more thereof. In embodiments, the compositions comprise an antibody compound described herein (including embodiments thereof) and a T cell, a natural killer cell, a monocyte, a neutrophil, a macrophage, or a combination of two or more thereof. In embodiments, the compositions comprise an antibody compound described herein (including embodiments thereof) and a T cell. In embodiments, the compositions comprise an antibody compound described herein (including embodiments thereof) and a CD4+ T cell, a CD8+ T cell, or a combination thereof. In embodiments, the compositions comprise an antibody compound described herein (including embodiments thereof) and a CD4+ T cell. In embodiments, the compositions comprise an antibody compound described herein (including embodiments thereof) and a CD8+ T cell. In embodiments, the compositions comprise an antibody compound described herein (including embodiments thereof) and a CD4+ T cell and a CD8+ T cell. In embodiments, the compositions comprise an antibody compound described herein (including embodiments thereof) and a B cell. In embodiments, the compositions comprise an antibody compound described herein (including embodiments thereof) and a natural killer cell. In embodiments, the compositions comprise an antibody compound described herein (including embodiments thereof) and a monocyte. In embodiments, the compositions comprise an antibody compound described herein (including embodiments thereof) and a macrophage. In embodiments, the compositions comprise an antibody compound described herein (including embodiments thereof) and a genetically-engineered immune cell. The skilled artisan will appreciate that the compositions can include the antibody bound to the immune cell.

In embodiments, the antibody compounds provided herein (including embodiments thereof) are bound to immune cells. In embodiments, the antibody compounds are non-covalently bound to immune cells. In embodiments, the antibody compounds are non-covalently bound to immune cell surface proteins, thereby forming antibody-coated immune cells. In embodiments, the antibody compounds are non-covalently bound to immune cell surface proteins via their Fc region, thereby forming antibody-coated immune cells. In embodiments, the immune cells are T cells, NK cells, monocytes, neutrophils, macrophages, or genetically-engineered immune cells. In embodiments, the immune cells are T cells. In embodiments, the immune cells are CD4+ T cells or CD8+ T cells. In embodiments, the immune cells are CD4+ T cells. In embodiments, the immune cells are CD8+ T cells. In embodiments, the T cells are human T cells. In embodiments, the immune cells are NK cells. In embodiments, the immune cells are monocytes. In embodiments, the immune cells are neutrophils. In embodiments, the immune cells are macrophages. In embodiments, the immune cells are genetically-engineered immune cells. In embodiments, the genetically-engineered immune cells are CAR T-cells.

Provided herein are compositions comprising an antibody compound described herein (including embodiments thereof), an immune cell, and an immune cell bound to an antibody compound. In embodiments, the immune cell is a T cell, a B cell, a natural killer cell, a monocyte, a neutrophil, a macrophage, a genetically-engineered immune cell, or a combination of two or more thereof. In embodiments, the compositions comprise an antibody compound described herein (including embodiments thereof), a T cell, and a T cell bound to an antibody compound. In embodiments, the compositions comprise an antibody compound described herein (including embodiments thereof), a T cell, and a T cell bound to an antibody compound; wherein the T cell is a CD4+ T cell, a CD8+ T cell, or a combination thereof. In embodiments, the compositions comprise an antibody compound described herein (including embodiments thereof), a CD4+ T cell, and a CD4+ T cell bound to an antibody compound. In embodiments, the compositions comprise an antibody compound described herein (including embodiments thereof), a CD8+ T cell, and a CD8+ T cell bound to an antibody compound. In embodiments, the compositions comprise an antibody compound described herein (including embodiments thereof), a CD4+ T cell, a CD8+ T cell, a CD4+ T cell bound to an antibody compound, and a CD8+ T cell bound to an antibody compound. In embodiments, the compositions comprise an antibody compound described herein (including embodiments thereof), a B cell, and a B cell bound to an antibody compound. In embodiments, the compositions comprise an antibody compound described herein (including embodiments thereof), a natural killer cell, and a natural killer cell bound to an antibody compound. In embodiments, the compositions comprise an antibody compound described herein (including embodiments thereof), a monocyte, and a monocyte bound to an antibody compound. In embodiments, the compositions comprise an antibody compound described herein (including embodiments thereof), a macrophage, and a macrophage bound to an antibody compound. In embodiments, the compositions comprise an antibody compound described herein (including embodiments thereof), a genetically-engineered immune cell, and a genetically-engineered immune cell bound to an antibody compound. In embodiments, the immune cell is non-covalently bound to the antibody compound.

In embodiments, the immune cells are activated. Methods of activating immune cells (e.g., T cells) are well-known in the art. In embodiments, immune calls are activated by contacting the immune cell receptor with anti-CD3 antibodies In embodiments, immune calls are activated by crosslinking the immune cell receptor with anti-CD3 antibodies. In embodiments, activated immune cells bound to the disclosed antibody compounds show increased specific cytotoxicity against target cancer cells. In embodiments, the target cancer cells are multiple myeloma cells. In embodiments, the multiple myeloma cells express CS-1. In embodiments, activated immune cells bound to the disclosed antibody compounds increase neutrophil infiltration into the cancer cells.

In embodiments, an “activated immune cell” is an immune cell that, inter alia, has an increased expression of cytokines and/or lytic enzymes. In embodiments, an “activated immune cell” is an immune cell that has an increased expression of IFN-γ, TNF-α, IL-2, GM-CSF, IL-8, or a combination of two or more thereof. An “increased expression” is relative to a control and can be at least a 2-fold increase in expression, at least a 2.5-fold, at least a 3-fold, at least a 4-fold, or a least a 5-fold increase in expression. In embodiments, the activated immune cell is a T cell. In embodiments, the T cell is a CD4+ T cell, a CD8+ T cell, or a combination thereof. In embodiments, the T cell is a CD4+ T cell. In embodiments, the T cell is a CD8+ T cell. In embodiments, the T cell is a combination of a CD4+ T cell and a CD8+ T cell. In embodiments, the activated immune cell is a B cell, a natural killer cell, a monocyte, a neutrophil, a macrophage, or a genetically-engineered immune cell. In embodiments, the activated immune cell is a B cell, a natural killer cell, a monocyte, a neutrophil, or a macrophage.

In embodiments, activated immune cells are prepared by a process comprising contacting an antibody compound described herein with an immune cell, thereby producing an activated immune cell. In embodiments, the activated immune cells are prepared by a process comprising contacting an immune cell with a compound comprising elotuzumab covalently bound to an OKT3 antibody, thereby producing an activated immune cell. In embodiments, the activated immune cells are prepared by a process comprising contacting an immune cell with a compound comprising an elotuzumab F(ab′)₂ fragment covalently bound to OKT3 antibody F(ab′)₂ fragment, thereby producing an activated immune cell. In embodiments, the activated immune cells are prepared by a process comprising contacting an immune cell with a compound comprising an elotuzumab F(ab′) fragment covalently bound to OKT3 antibody F(ab′) fragment, thereby producing an activated immune cell. In embodiments, the activated immune cells are prepared ex vivo. In embodiments, the activated immune cells are prepared in vitro. In embodiments, the activated immune cells are prepared in vivo.

### Methods

Provided herein are methods of treating a cancer in a patient in need thereof comprising administering to the patient an effective amount of the antibody compound described herein (including embodiments thereof) to treat cancer. Provided herein are methods of treating multiple myeloma in a patient in need thereof comprising administering to the patient an effective amount of the antibody compound described herein (including embodiments thereof) to treat multiple myeloma. In embodiments, the antibody compounds comprise elotuzumab covalently bound to an OKT3 antibody. In embodiments, the antibody compounds comprise a cysteine residue in elotuzumab covalently bound to a cysteine residue in an OKT3 antibody. In embodiments, the cysteine residue is in the hinge region. In embodiments, the antibody compounds comprise the cysteine hinge region of elotuzumab covalently bound to the cysteine hinge region of an OKT3 antibody. In embodiments, the antibody compounds comprise an elotuzumab F(ab′)₂ fragment covalently bound to OKT3 antibody F(ab′)₂ fragment. In embodiments, the antibody compounds comprise a cysteine residue in an elotuzumab F(ab′)₂ fragment covalently bound to a cysteine residue in an OKT3 antibody F(ab′)₂ fragment. In embodiments, the cysteine residue is in the hinge region. In embodiments, the antibody compounds comprise the cysteine hinge region of an elotuzumab F(ab′)₂ fragment covalently bound to the cysteine hinge region of an OKT3 antibody F(ab′)₂ fragment. In embodiments, the antibody compounds comprise an elotuzumab F(ab′) fragment covalently bound to OKT3 antibody F(ab′) fragment. In embodiments, the antibody compounds comprise a cysteine residue in an elotuzumab F(ab′) fragment covalently bound to a cysteine residue in an OKT3 antibody F(ab′) fragment. In embodiments, the cysteine residue is in the hinge region. In embodiments, the antibody compounds comprise the cysteine hinge region of an elotuzumab F(ab′) fragment covalently bound to the cysteine hinge region of an OKT3 antibody F(ab′) fragment. In embodiments, the OKT3 antibody is a humanized OKT3 antibody. In embodiments, the compounds comprise: (i) an elotuzumab antibody, an elotuzumab F(ab′)₂ fragment, or an elotuzumab F(ab′) fragment, and (ii) a humanized OKT3 antibody, a humanized OKT3 antibody F(ab′)₂ fragment, or a humanized OKT3 antibody F(ab′) fragment; wherein the cysteine hinge region of (i) is covalently bonded to the cysteine hinge region of (ii). In embodiments, the compounds comprise: (i) an elotuzumab antibody, an elotuzumab F(ab′)₂ fragment, or an elotuzumab F(ab′) fragment, and (ii) a humanized OKT3 antibody, a humanized OKT3 antibody F(ab′)₂ fragment, or a humanized OKT3 antibody F(ab′) fragment; wherein a cysteine residue in (i) is covalently bonded to a cysteine residue in (ii). In embodiments, a cysteine residue is in the hinge region of (i) and a cysteine residue is in the hinge region of (ii).

In embodiments, the method for treating cancer in a patient in need thereof comprising obtaining a biological sample from the patient, wherein the biological sample comprises immune cells; contacting the biological sample with the antibody compounds described herein (including embodiments thereof) to produce activated immune cells bound to the antibody compound; and administering to the patient an effective amount of the activated immune cells bound to the antibody compound. In embodiments, the immune cells are isolated from the biological sample prior to contacting the immune cells with the antibody compounds described herein. In embodiments, the method for treating cancer in a patient in need thereof comprises obtaining a biological sample from the patient, wherein the biological sample comprises immune cells; isolating the immune cells from the biological sample; contacting the isolated immune cells with the antibody compounds described herein (including embodiments thereof) to produce activated immune cells bound to the antibody compound; and administering to the patient an effective amount of the activated immune cells bound to the antibody compound. In embodiments, the method for treating cancer in a patient in need thereof comprises obtaining immune cells from the patient; contacting the immune cells with the antibody compounds described herein (including embodiments thereof) to produce activated immune cells bound to the antibody compound; and administering to the patient an effective amount of the activated immune cells bound to the antibody compound.

In embodiments, the immune cells obtained from the patient are any immune cell described herein. In embodiments, the immune cells are T cells, B cells, natural killer cells, monocytes, neutrophils, macrophages, or genetically-engineered immune cells. In embodiments, the immune cells are human peripheral blood mononuclear cells (e.g., T cells, B cells, natural killer cells). In embodiments, the immune cells are T cells. In embodiments, the immune cells are activated with anti-CD3 antibody and IL-2, and then expanded in vitro for a period up to 30 days prior to binding the immune cells to the antibody compounds described herein. In embodiments, the activated immune cells are expanded for 1 to 30 days. In embodiments, the activated immune cells are expanded for 1 to 25 days. In embodiments, the activated immune cells are expanded for 1 to 20 days. In embodiments, the activated immune cells are expanded for 1 to 15 days. In embodiments, the activated immune cells are expanded for 1 to 10 days. In embodiments, the activated immune cells are expanded for 1 to 7 days. In embodiments, the activated immune cells are expanded for 1 to 6 days. In embodiments, the activated immune cells are expanded for 1 to 5 days. In embodiments, the activated immune cells are expanded for 1 to 4 days. In embodiments, the activated immune cells are expanded for 1 to 3 days. In embodiments, the activated immune cells are expanded for 1 to 2 days. In embodiments, the activated immune cells are expanded for 2 to 7 days. In embodiments, the activated immune cells are expanded for 2 to 6 days. In embodiments, the activated immune cells are expanded for 2 to 5 days. In embodiments, the activated immune cells are expanded for 2 to 4 days. In embodiments, the activated immune cells are expanded for 2 to 3 days. In embodiments, the activated immune cells are expanded for 3 to 7 days. In embodiments, the activated immune cells are expanded for 3 to 6 days. In embodiments, the activated immune cells are expanded for 3 to 5 days. In embodiments, the activated immune cells are expanded for 3 to 4 days. In embodiments, the activated immune cells are expanded for 4 to 7 days. In embodiments, the activated immune cells are expanded for 4 to 6 days. In embodiments, the activated immune cells are expanded for 4 to 5 days. In embodiments, the activated immune cells are expanded for 5 to 7 days. In embodiments, the activated immune cells are expanded for 5 to 6 days. In embodiments, the activated immune cells are expanded for 6 to 7 days. In embodiments, the activated immune cells are expanded for 1 day. In embodiments, the activated immune cells are expanded for 2 days. In embodiments, the activated immune cells are expanded for 3 days. In embodiments, the activated immune cells are expanded for 4 days. In embodiments, the activated immune cells are expanded for 5 days. In embodiments, the activated immune cells are expanded for 6 days. In embodiments, the activated immune cells are expanded for 7 days.

In embodiments, the method for treating cancer in a patient in need thereof comprising obtaining a biological sample from the patient, wherein the biological sample comprises immune cells; contacting the biological sample with anti-CD3 antibody and IL-2; and then contacting the biological sample with the antibody compounds described herein (including embodiments thereof) to produce activated immune cells bound to the antibody compound; and administering to the patient an effective amount of the activated immune cells bound to the antibody compound. In embodiments, the immune cells are isolated from the biological sample prior to contacting the immune cells with the antibody compounds described herein. In embodiments, the method for treating cancer in a patient in need thereof comprises obtaining a biological sample from the patient, wherein the biological sample comprises immune cells; isolating the immune cells from the biological sample; contacting the isolated immune cells with anti-CD3 antibody and IL-2; contacting the isolated immune cells with the antibody compounds described herein (including embodiments thereof) to produce activated immune cells bound to the antibody compound; and administering to the patient an effective amount of the activated immune cells bound to the antibody compound. In embodiments, the method for treating cancer in a patient in need thereof comprises obtaining immune cells from the patient; contacting the immune cells with anti-CD3 antibody and IL-2; contacting the immune cells with the antibody compounds described herein (including embodiments thereof) to produce activated immune cells bound to the antibody compound; and administering to the patient an effective amount of the activated immune cells bound to the antibody compound.

In embodiments, the method for treating cancer in a patient in need thereof comprising obtaining a biological sample from the patient, wherein the biological sample comprises immune cells; contacting the biological sample with anti-CD3 antibody and IL-2; expanding the immune cells; contacting the biological sample with the antibody compounds described herein (including embodiments thereof) to produce activated immune cells bound to the antibody compound; and administering to the patient an effective amount of the activated immune cells bound to the antibody compound. In embodiments, the immune cells are isolated from the biological sample prior to contacting the immune cells with the antibody compounds described herein. In embodiments, the method for treating cancer in a patient in need thereof comprises obtaining a biological sample from the patient, wherein the biological sample comprises immune cells; isolating the immune cells from the biological sample; contacting the isolated immune cells with anti-CD3 antibody and IL-2; expanding the isolated immune cells; contacting the isolated immune cells with the antibody compounds described herein (including embodiments thereof) to produce activated immune cells bound to the antibody compound; and administering to the patient an effective amount of the activated immune cells bound to the antibody compound. In embodiments, the method for treating cancer in a patient in need thereof comprises obtaining immune cells from the patient; contacting the immune cells with anti-CD3 antibody and IL-2; expanding the immune cells; contacting the immune cells with the antibody compounds described herein (including embodiments thereof) to produce activated immune cells bound to the antibody compound; and administering to the patient an effective amount of the activated immune cells bound to the antibody compound.

In embodiments, the antibody compounds described herein are non-covalently attached to the immune cells. In embodiments, the antibody compounds described herein are non-covalently attached to the immune cells by binding to the immune cell surface proteins via their Fc region. In embodiments, the non-covalent binding of the antibody compounds described herein to the immune cell surface proteins produces antibody compound-coated immune cells. In embodiments, activated immune cells bound to the disclosed antibody compounds have increased specific cytotoxicity against target cancer cells. In embodiments, the target cancer cells are multiple myeloma cells. In embodiments, the multiple myeloma cells express CS-1. In embodiments, activated immune cells bound to the antibody compounds described herein increase neutrophil infiltration into the cancer cells.

### Humanized OKT3 Antibody

Provided herein are humanized OKT3 antibodies. In embodiments, the humanized OKT3 antibody is a humanized OKT3 antibody F(ab′) fragment. In embodiments, the humanized OKT3 antibody is a humanized OKT3 antibody F(ab′)₂ fragment. In embodiments, the humanized OKT3 antibody is a single chain variable domain (scFv).

In embodiments, the humanized OKT3 antibody comprises a light chain variable domain comprising a CDR L1 having SEQ ID NO:1, a CDR L2 having SEQ ID NO:2 and a CDR L3 having SEQ ID NO:3; and a heavy chain variable domain comprising a CDR H1 having SEQ ID NO:4, a CDR H2 having SEQ ID NO:5, and a CDR H3 having SEQ ID NO:6.

In embodiments, the humanized OKT3 antibody comprises a light chain variable domain comprising a CDR L1 which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:1, a CDR L2 which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:2 and a CDR L3 which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:3; and a heavy chain variable domain comprising a CDR H1 which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:4, a CDR H2 which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:5, and a CDR H3 which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:6.

In embodiments, the humanized OKT3 antibody comprises a light chain variable domain having SEQ ID NO:9, and a heavy chain variable domain having SEQ ID NO:10.

In embodiments, the light chain variable domain of the humanized OKT3 antibody comprises a sequence is at least 80%, at least 85%, at least 90%, at least 91%, at least 92%, at least 93%, at least 94%, at least 95%, at least 96%, at least 97%, at least 98%, at least 99%, or 100% identical to SEQ ID NO:9. In embodiments, the light chain variable domain of the humanized OKT3 antibody comprises a sequence that is at least 80% identical to SEQ ID NO:9. In embodiments, the light chain variable domain of the humanized OKT3 antibody comprises a sequence that is at least 85% identical to SEQ ID NO:9. In embodiments, the light chain variable domain of the humanized OKT3 antibody comprises a sequence that is at least 90% identical to SEQ ID NO:9. In embodiments, the light chain variable domain of the humanized OKT3 antibody comprises a sequence that is at least 95% identical to SEQ ID NO:9. In embodiments, the light chain variable domain of the humanized OKT3 antibody comprises a sequence that is at least 98% identical to SEQ ID NO:9. In embodiments, the light chain variable domain of the humanized OKT3 antibody comprises a sequence that is at least 99% identical to SEQ ID NO:9. In embodiments, the light chain variable domain of the humanized OKT3 antibody comprises a sequence having SEQ ID NO:9. In embodiments where the light chain variable domain has less than 100% identity to SEQ ID NO:9, the light chain variable domain has 100% sequence identity to the CDRs within the light chain variable domain.

In embodiments, the heavy chain variable domain of the humanized OKT3 antibody comprises a sequence is at least 80%, at least 85%, at least 90%, at least 91%, at least 92%, at least 93%, at least 94%, at least 95%, at least 96%, at least 97%, at least 98%, at least 99%, or 100% identical to SEQ ID NO:10. In embodiments, the heavy chain variable domain of the humanized OKT3 antibody comprises a sequence that is at least 80% identical to SEQ ID NO:10. In embodiments, the heavy chain variable domain of the humanized OKT3 antibody comprises a sequence that is at least 85% identical to SEQ ID NO:10. In embodiments, the heavy chain variable domain of the humanized OKT3 antibody comprises a sequence that is at least 90% identical to SEQ ID NO:10. In embodiments, the heavy chain variable domain of the humanized OKT3 antibody comprises a sequence that is at least 95% identical to SEQ ID NO:10. In embodiments, the heavy chain variable domain of the humanized OKT3 antibody comprises a sequence that is at least 98% identical to SEQ ID NO:10. In embodiments, the heavy chain variable domain of the humanized OKT3 antibody comprises a sequence that is at least 99% identical to SEQ ID NO:10. In embodiments, the heavy chain variable domain of the humanized OKT3 antibody comprises a sequence having SEQ ID NO:10. In embodiments where the heavy chain variable domain has less than 100% identity to SEQ ID NO:10, the heavy chain variable domain has 100% sequence identity to the CDRs within the heavy chain variable domain.

In embodiments, the light chain variable domain of the humanized OKT3 antibody comprises a signal sequence which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:7. In embodiments, the light chain variable domain of the humanized OKT3 antibody comprises a signal sequence which is at least 90% identical to SEQ ID NO:7. In embodiments, the light chain variable domain of the humanized OKT3 antibody comprises a signal sequence which is at least 95% identical to SEQ ID NO:7. In embodiments, the light chain variable domain of the humanized OKT3 antibody comprises a signal sequence which is at least 98% identical to SEQ ID NO:7. In embodiments, the light chain variable domain of the humanized OKT3 antibody comprises a signal sequence which is at least 99% identical to SEQ ID NO:7. In embodiments, the light chain variable domain of the humanized OKT3 antibody comprises a signal sequence having SEQ ID NO:7.

In embodiments, the heavy chain variable domain of the humanized OKT3 antibody comprises a signal sequence which is at least 80%, at least 85%, at least 90%, at least 91%, at least 92%, at least 93%, at least 94%, at least 95%, at least 96%, at least 97%, at least 98%, at least 99%, or 100% identical to SEQ ID NO:8. In embodiments, the heavy chain variable domain of the humanized OKT3 antibody comprises a signal sequence which is at least 80% identical to SEQ ID NO:8. In embodiments, the heavy chain variable domain of the humanized OKT3 antibody comprises a signal sequence which is at least 85% identical to SEQ ID NO:8. In embodiments, the heavy chain variable domain of the humanized OKT3 antibody comprises a signal sequence which is at least 90% identical to SEQ ID NO:8. In embodiments, the heavy chain variable domain of the humanized OKT3 antibody comprises a signal sequence which is at least 95% identical to SEQ ID NO:8. In embodiments, the heavy chain variable domain of the humanized OKT3 antibody comprises a signal sequence which is at least 98% identical to SEQ ID NO:8. In embodiments, the heavy chain variable domain of the humanized OKT3 antibody comprises a signal sequence which is at least 99% identical to SEQ ID NO:8. In embodiments, the heavy chain variable domain of the humanized OKT3 antibody comprises a signal sequence having SEQ ID NO:8.

Provided herein are methods of treating cancer in a patient in need thereof comprising administering to the patient an effective amount of the humanized OKT3 antibodies described herein (including embodiments thereof) to treat cancer. In embodiments, the methods comprise treating multiple myeloma in a patient in need thereof comprising administering to the patient an effective amount of the humanized OKT3 antibodies described herein (including embodiments thereof) to treat multiple myeloma. In embodiments, the humanized OKT3 antibody is a humanized OKT3 antibody F(ab′) fragment. In embodiments, the humanized OKT3 antibody is a humanized OKT3 antibody F(ab′)₂ fragment.

### Pharmaceutical Compositions

Any of the antibody and antibody compounds described herein may be administered to a subject in a pharmaceutical composition further comprising a pharmaceutically acceptable excipient. The compositions are suitable for formulation and administration in vitro or in vivo. Suitable carriers and excipients and their formulations are known in the art and described, e.g., Remington: The Science and Practice of Pharmacy, 21st Ed, Lippicott Williams & Wilkins (2005). Provided herein are pharmaceutical compositions comprising the antibody compounds described herein (including embodiments thereof) and a pharmaceutically acceptable excipient. Provided herein are pharmaceutical compositions comprising the humanized OKT3 antibodies described herein (including embodiments thereof) and a pharmaceutically acceptable excipient. Provided herein are pharmaceutical compositions comprising the activated immune cells described herein (including embodiments thereof) and a pharmaceutically acceptable excipient. Provided herein are pharmaceutical compositions comprising the activated immune cells bound to the antibody compounds described herein (including embodiments thereof) and a pharmaceutically acceptable excipient.

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful. Pharmaceutically acceptable excipients can be used in pharmaceutical compositions for therapeutic purposes (e.g., treating a disease) and/or diagnostic purposes (e.g., imaging, such as positron emission tomography).

Solutions of the pharmaceutical compositions can be prepared in water suitably mixed with a lipid or surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms. Solutions can be administered, e.g., parenterally, such as subcutaneously or intravenously (e.g., infusion or bolus).

Pharmaceutical compositions can be delivered via intranasal or inhalable solutions. The intranasal composition can be a spray, aerosol, or inhalant. The inhalable composition can be a spray, aerosol, or inhalant. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known in the art.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In aspects, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may be between about 1 to about 75% of the weight of the unit. The amount of nucleic acids in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the recombinant proteins in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for rapid penetration, delivering high concentrations of the active agents to a small area.

### Dose and Dosing Regimens

The dosage and frequency (single or multiple doses) of the compositions, antibodies, antibody compounds, and immune cells bound to antibody compounds described herein administered to a subject can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods, antibodies, and compounds described herein. Adjustment and manipulation of established dosages (e.g., frequency and duration) are within the ability of the skilled artisan.

For any composition, antibody, antibody compound, activated immune cells, or immune cell bound to antibody compound described herein, the effective amount can be initially determined from cell culture assays. Target concentrations will be those concentrations that are capable of achieving the methods described herein, as measured using the methods described herein or known in the art. As is known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the compositions, antibodies, antibody compounds, activated immune cells, or immune cells bound to antibody compounds described herein can be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Dosage amounts and intervals can be adjusted individually to provide levels of the recombinant proteins effective for the particular clinical indication being treated. This will provide a therapeutic regimen that is commensurate with the severity of the individual's disease state.

Utilizing the teachings provided herein, an effective therapeutic treatment regimen can be planned that does not cause substantial toxicity and yet is effective to treat the clinical disease or symptoms demonstrated by the patient. This planning should involve the careful choice of compositions, antibodies, antibody compounds, activated immune cells, or immune cells bound to antibody compounds by considering factors such as compound potency, relative bioavailability, patient body weight, presence and severity of adverse side effects.

In embodiments, the compositions, antibodies, antibody compounds, activated immune cells, or immune cells bound to antibody compounds described herein are administered to a patient at an amount of about 0.0001 mg/kg to about 500 mg/kg. In embodiments, the compositions, antibodies, antibody compounds, activated immune cells, or immune cells bound to antibody compounds are administered to a patient in an amount of about 0.0001 mg/kg, 0.001 mg/kg, 0.01 mg/kg, 0.1 mg/kg, 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, or 300 mg/kg. It is understood that where the amount is referred to as “mg/kg,” the amount is milligram per kilogram body weight of the patient. In embodiments, the compositions, antibodies, antibody compounds, activated immune cells, or immune cells bound to antibody compounds described herein are administered to a patient in an amount from about 0.0001 mg to about 500 mg per day.

### EXAMPLES

Bispecific T cell engaging antibodies (bsAbs) have emerged as novel and powerful therapeutic agents for redirecting T cells towards antigen-specific tumor killing. The cell surface glycoprotein and SLAM family member, CS1, exhibits stable and high-level expression on malignant plasma cells including multiple myeloma, which is indicative of an ideal target for bsAb therapy.

The inventors developed a CS1 bsAb (CS1-dbBiTE) using click chemistry to conjugate intact anti-CS1 antibody (elotuzumab) and anti-huOKT3 antibody at their respective hinge regions. Using a cellular therapy approach, human T cells were armed ex-vivo with CS1-dbBiTE prior to examining effector activity.

The data shows that arming T cells with CS1-dbBiTE induced T cell activation and expansion and subsequent cytotoxic activity against CS1-bearing MM tumors, demonstrated by significant CD107a expression as well as inflammatory cytokine secretion. CS1-dbBiTE armed T cells showed significantly reduced effector activity in the absence of CS1 expression. Similarly, in MM mouse xenograft studies, armed T cells exhibited effective anti-tumor efficacy highlighted by reduced tumor burden in MM.1S tumor bearing mice compared to controls. On the basis of these findings, the rationale for CS1 targeting by human T cells armed with CS1-dbBiTE presents an effective therapeutic approach for targeting MM.

### Example 1

The humanized anti-CD3 mAb was derived by CDR grafting the murine OKT3 hybridoma named gOKT3-5 (Adair, J. R. et al, 1994 Hum Antibod Hybridomas). The genes encoding the gOKT3-7 variant, composed of the gLC and gHG, were synthesized with human IgG1/k immunoglobulin constant domains, cloned into the pEE12/6 glutamine synthetase vector (Lonza Biologics, Cambridge, UK) and transiently expressed in EXPI293 cells (Thermo Fisher Scientific, Waltham, MA). The secreted antibody was purified by Protein rA (Millipore Sigma, St Louis, MO) and ceramic hydroxyapatite chromatography (Bio-Rad Laboratories, Hercules, CA).

### Example 2

### Generation of CS1-dbBiTEs and Binding to Positive Targets

dbBiTEs are bispecific antibodies in which a tumor antigen targeting antibody is crosslinked by Click chemistry to an anti-CD3 antibody. (Ref. 22). In this study, the starting antibodies were anti-CS1 (Elotuzumab) and a humanized version of anti-CD3 (huOKT3) as previously described by Kujawski et al, BMC Cancer, 19:882-882 (2019). Both antibodies are intact IgG1s that have been modified by Click reagents in their reduced hinge regions. After clicking together, the dbBiTEs are purified by size exclusion chromatography to separate the unreacted 150 kDa antibodies from their 300 kDa clicked product. The resulting dbBiTE exhibits a major band at 300 kDa on non-reducing SDS gels (FIG. 1A). A schematic showing details of the clicked products and their predicted binding to target and T cells is shown in FIG. 1B. Flow analysis of their actual binding to their cognate antigens, namely CS1 on MM.1S and CD3 on human T-cells is shown in (FIGS. 1C-1D). From this analysis the binding of the dbBiTE to MM.1S requires a higher concentration than that for binding to human T cells. This result emphasizes that elotuzumab is a lower affinity antibody compared to huOKT3. Thus, further studies were performed at a minimum concentration of 1 μg/mL of dbBiTE for coating T cells. (Ref 22).

### CS1-dbBiTE Armed T Cells Mediate Potent Cytotoxicity to MM

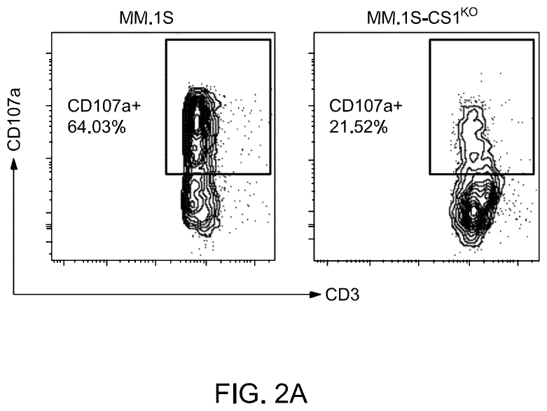

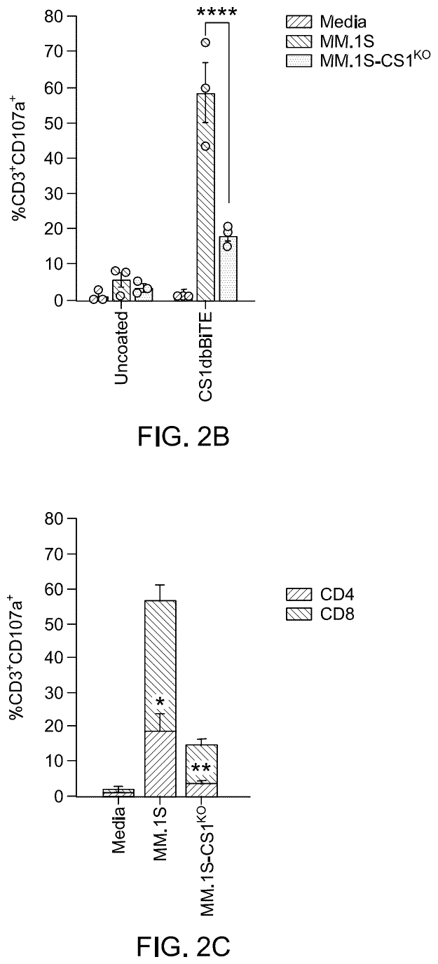

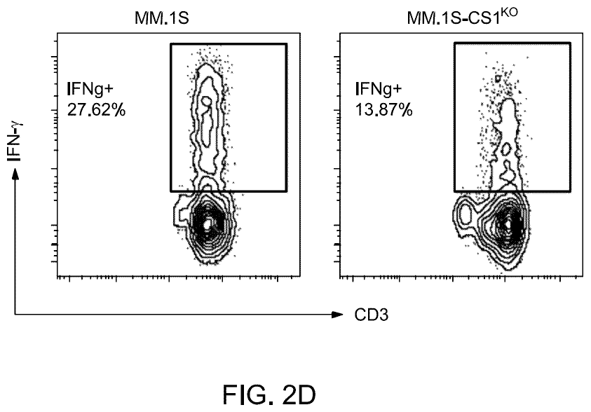

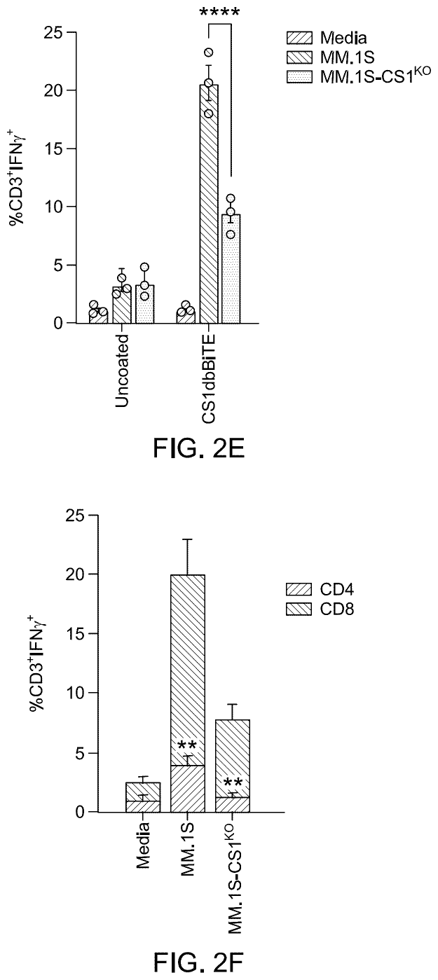

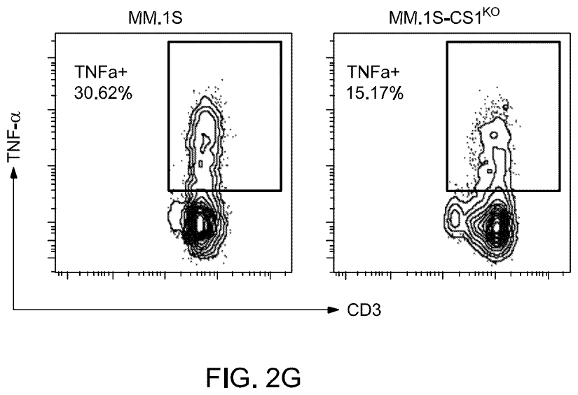

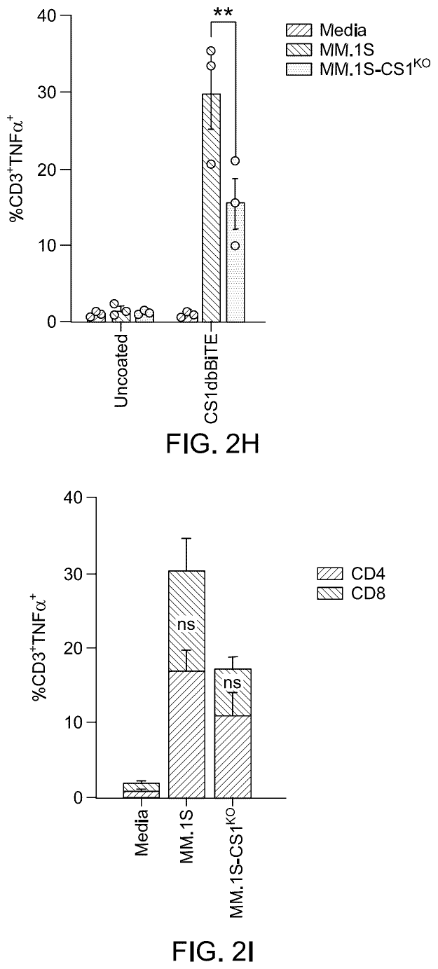

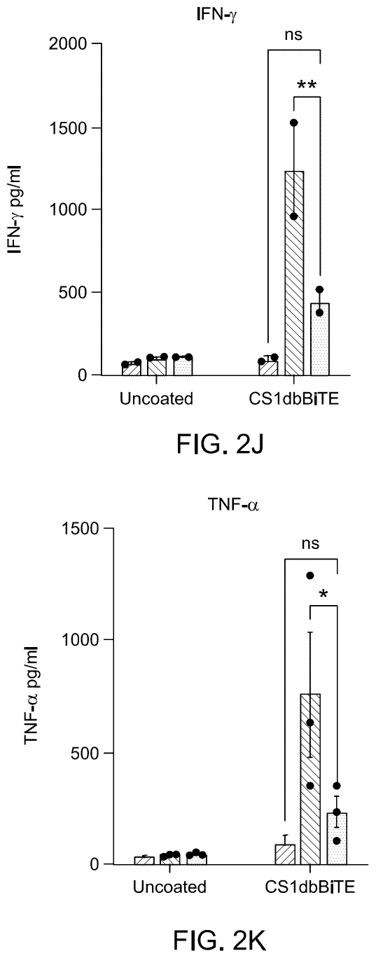

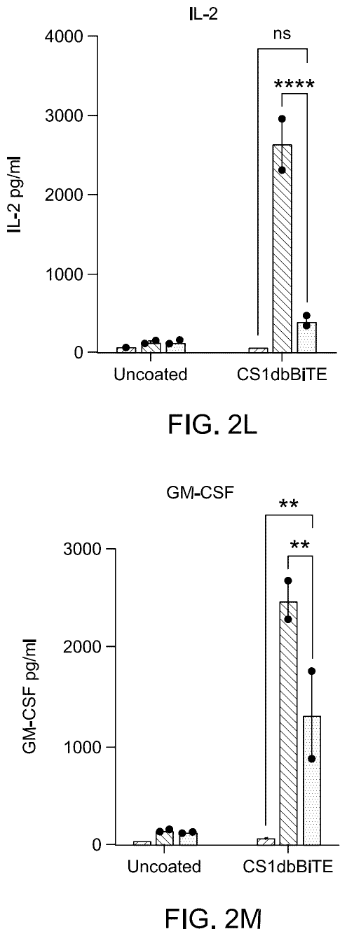

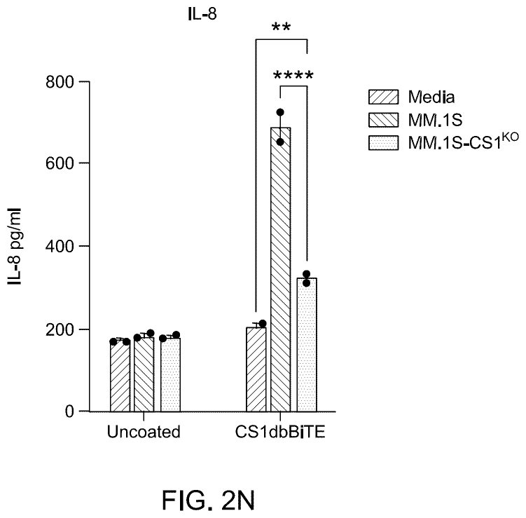

T cell engagement with tumor and subsequent activation through CD3 receptor signaling is essential for downstream tumor cell lysis. Following successful dbBiTE binding to positive targets, the in vitro cytotoxic response of dbBiTE-armed, activated human T cells (1 μg/ml per 10M cells) against multiple myeloma line MM.1S was examined. 1 μg/ml final dbBiTE concentration based on titration data was chosen because higher concentrations did not significantly impact effector cell responses (data not shown). In addition, a CS1 knockout MM.1S line (MM.1S-CS1^{KO}) was generated to ensure specificity of dbBiTE binding and tumor cell viability was not influenced by CS1 knockout. A significant CD107a (p=0.002) expression by armed T cells against target lines compared to unarmed controls (FIGS. 2A-2C) was observed. Furthermore, effector cell degranulation was specific for wild-type MM.1S as indicated by a significant reduction in CD107a expression (P=0.0021) in the absence of CS1-expressing targets. Examination of Th1 cytokine responses as indicators of effective cytolytic activity against tumor cells revealed more than 2-fold increase in IFN-γ and TNF-α positive cells detected by intracellular cytokine staining (FIGS. 2D-2I), when dbBiTE-armed T cells were co-cultured with MM.1S targets in comparison to MM.1S-CS1® lines. This observation was consistent with increases in IFN-γ and TNF-α (P<0.05) production, following analysis of co-culture supernatants (FIGS. 2D-2I). The T cell growth factors IL-2 and GM-CSF as well as the chemokine IL-8, were also significantly elevated (FIGS. 2J-2N), whereas no changes were seen in production of Th2 cytokines IL-4, IL-5, IL-6 and IL-10 following target engagement. It is noteworthy that degranulating as well as IFN-γ positive cells were predominantly CD8⁺ T cells whereas TNF-α positive cells were primarily CD4⁺ T cells although not significant when compared to CD8+ T cells.

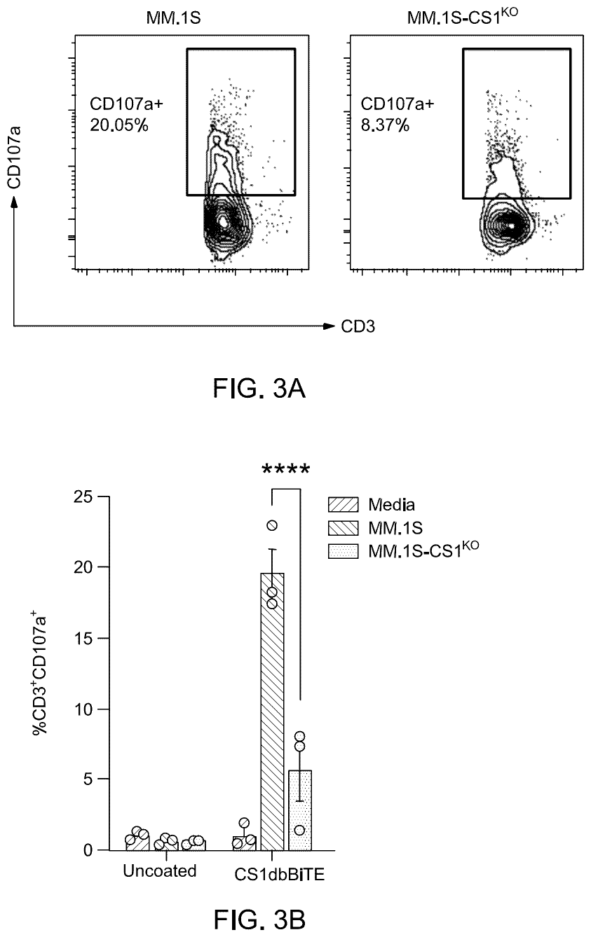

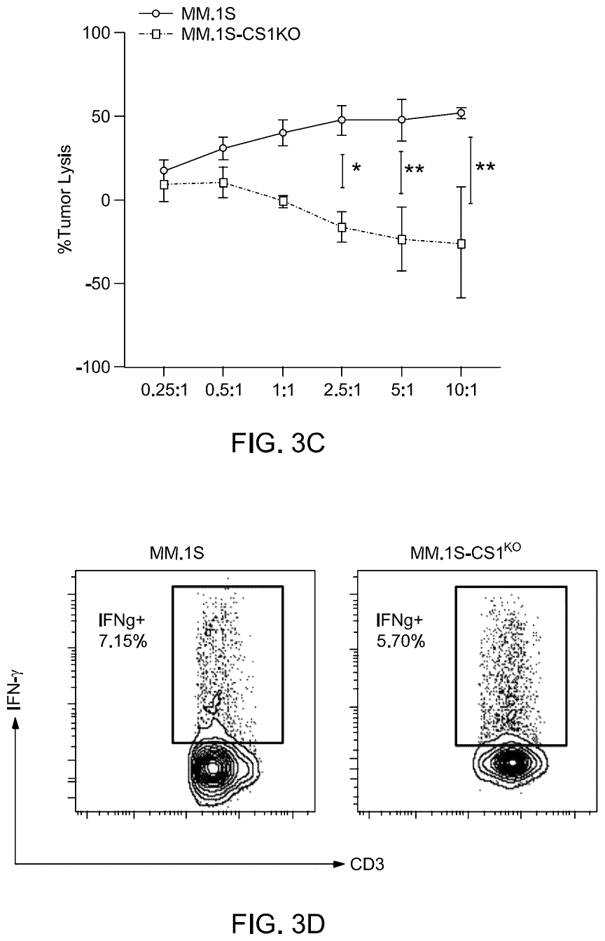

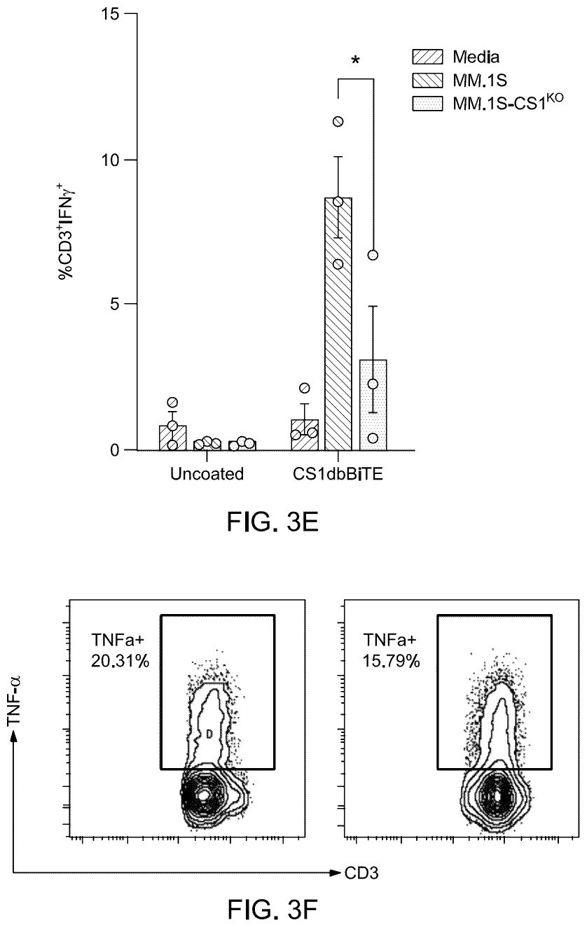

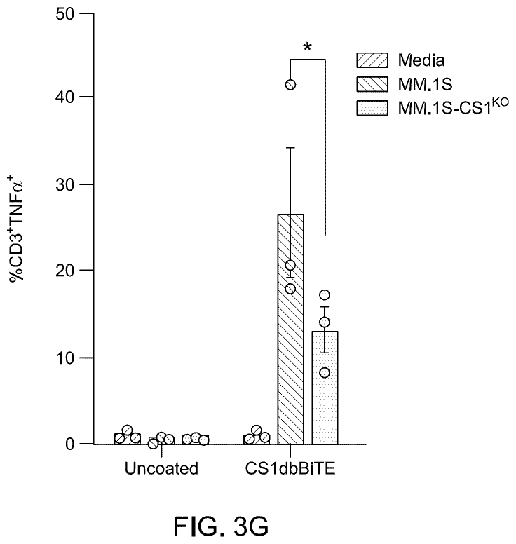

Given that bi-specific T cell engaging antibody therapy is achieved by direct infusion into patients without ex-vivo T cell activation, the potency of dbBiTE armed resting T cells (unstimulated) against target lines (wildtype MM.1S and MM.1S-CS1K) was investigated. Although armed resting cells demonstrated significant CD107a (FIGS. 3A-3B) and intracellular cytokine expression (FIGS. 3D-3G), the magnitude of effector responses was lower when compared to activated T cells, highlighting the significance of dbBiTE for cellular therapy approaches as well as for direct patient infusion in the clinic. Analysis of long-term killing by armed T cells revealed a dose-dependent lysis of wildtype MM.1S tumors at increasing effector-target (e:t) ratios from 0.25:1 to 10:1, whereas tumor lysis was absent in CS1 knockout lines (FIG. 3C). Similarly, resting T cells also demonstrated dose-dependent tumor lysis with maximum killing (˜50%) at 10:1 e:t ratio. In contrast to activated T cells, degranulating and intracellular cytokine positive, resting T cells were predominantly CD4⁺ cells.

### CS1-dbBiTE Induces T Cell Activation and Differentiation

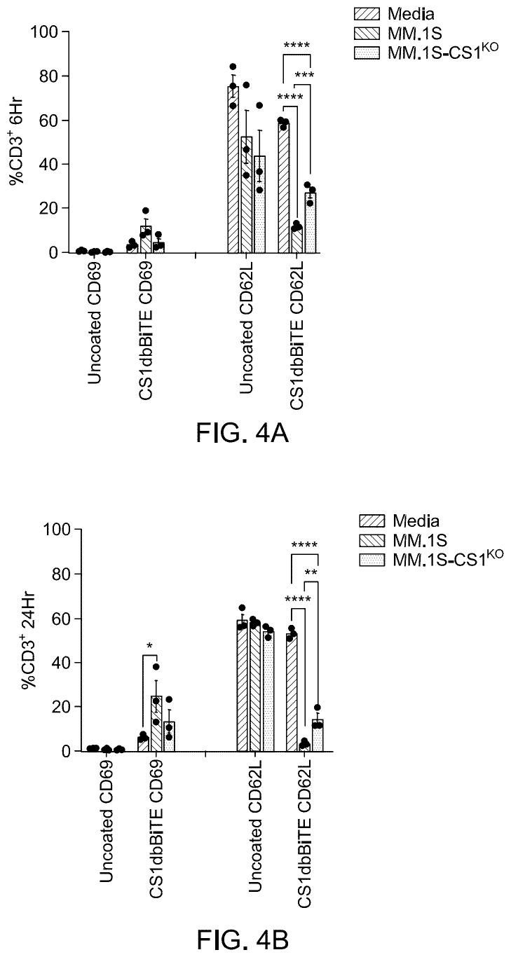

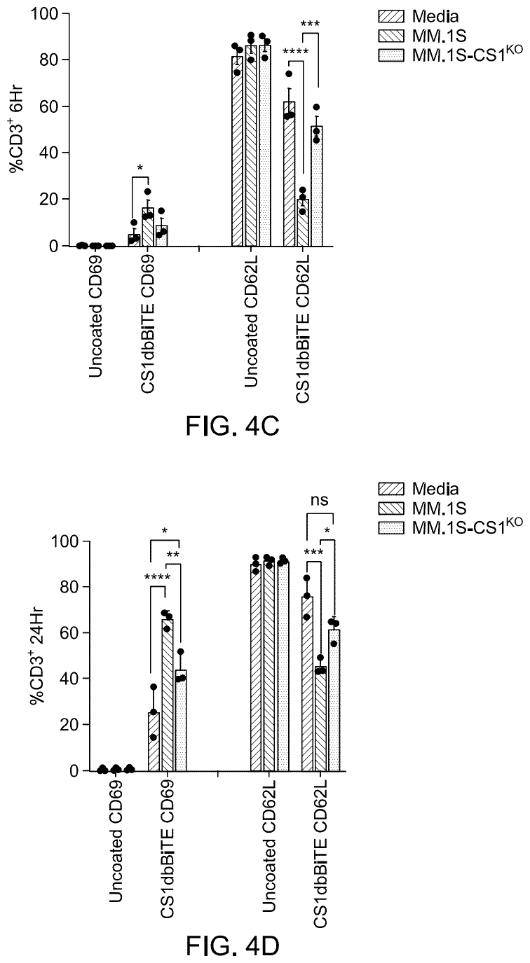

The inventors examined whether arming T cells with CS1-dbBiTE alone (in the absence of antigen) influenced T cell activation and memory marker expression and found an increase in expression of the early activation marker CD69, above baseline levels in both dbBiTE-armed, activated (FIGS. 4A-4B) and resting T cells (FIGS. 4C-4D) showing that arming T cells with dbBiTE may only promote T cell activation. Additionally, we observed a significantly higher CD69 expression in the presence of CS1 targets (antigen) after 24 hours of co-culture, with resting cells demonstrating greater than 40% increase in CD69 expression compared to about 10% increase in activated cells, which could presumably reflect the different activation states after two activation cycles (activated) vs. one activation (un-stimulated) cells. The absence of CD69 expression in unarmed but activated cells after 6- and 24-hours, following CD3/CD28 stimulation indicates timing is crucial for expression of CD69. Conversely, CD62L memory marker showed high expression in unarmed cells but decreased expression in the presence of CS1 targets following dbBiTE arming (FIGS. 4A-4D), highlighting the differentiation of T cells from a more central memory phenotype to effector memory populations.

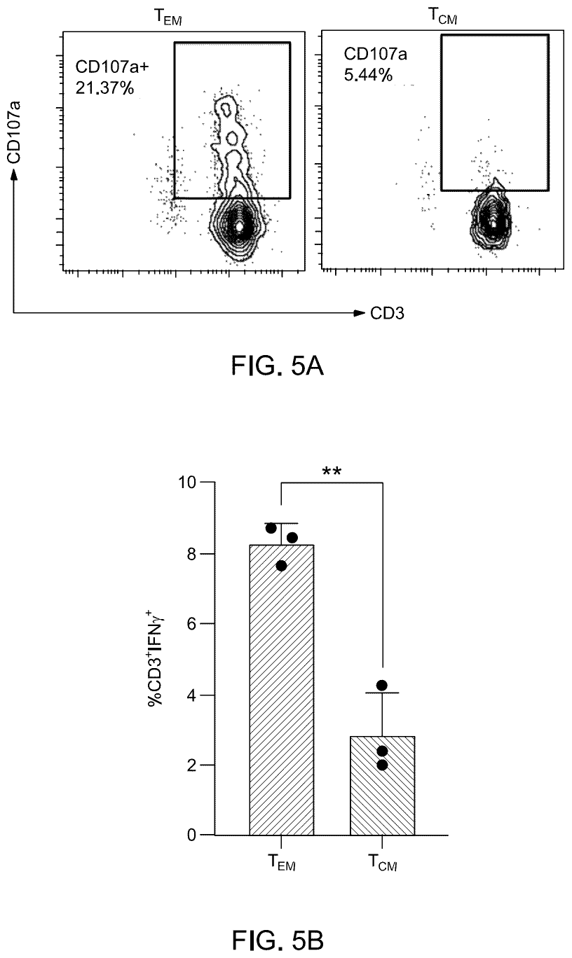

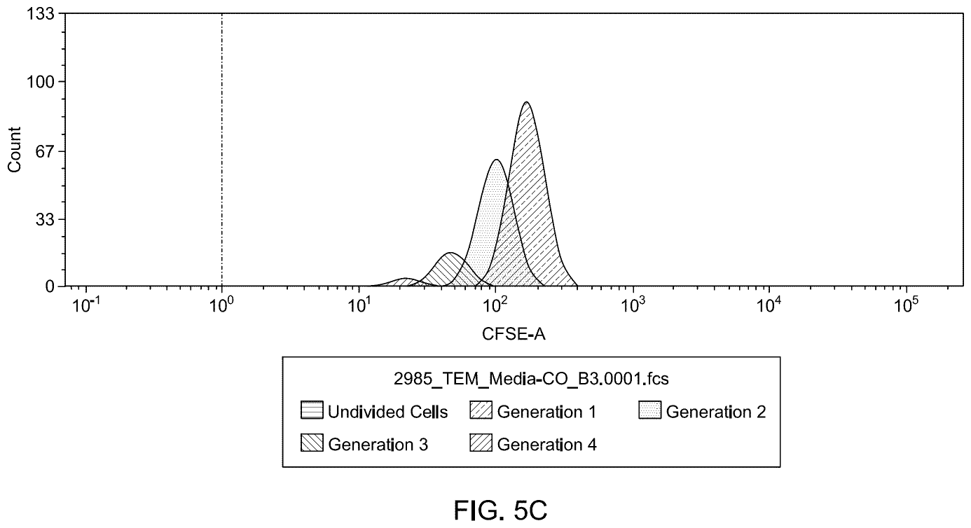

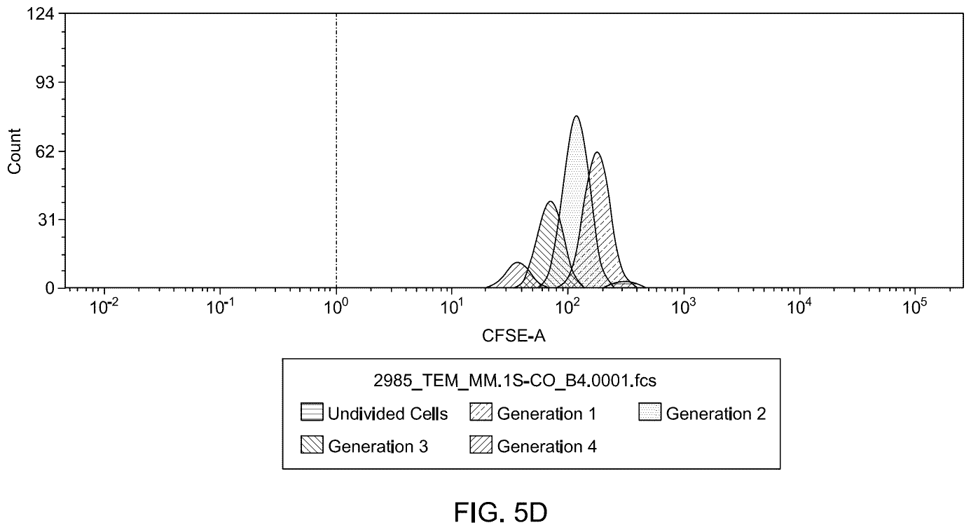

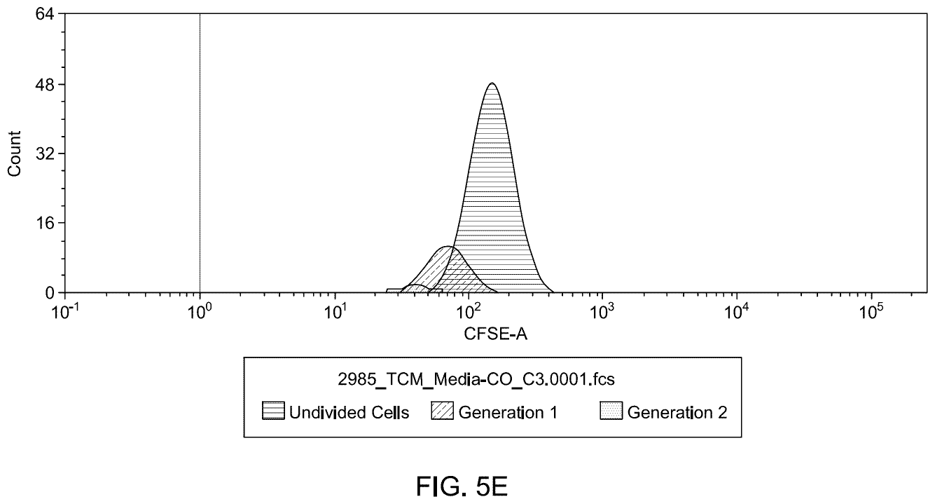

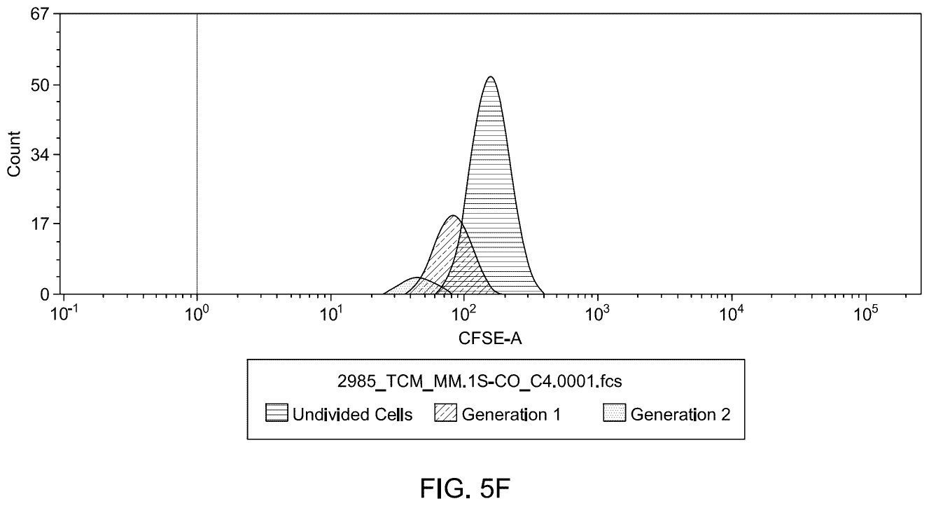

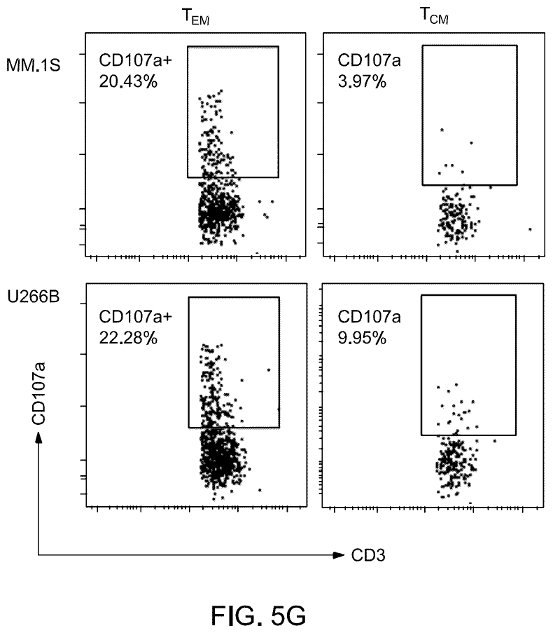

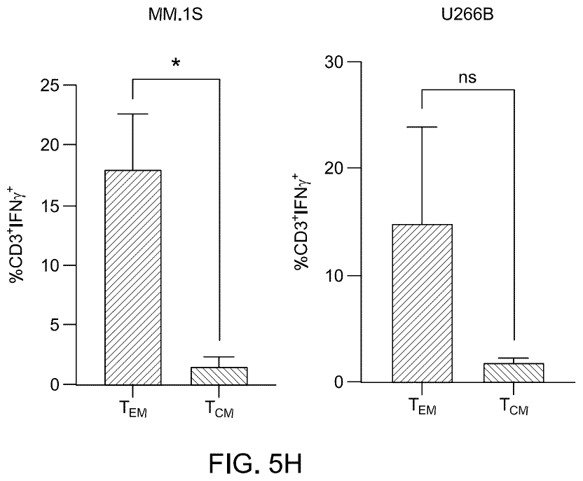

To determine the impact of T cell differentiation state on CS1-dbBiTE on activity, we performed T cell subset isolation from healthy donor PBMC and evaluated their cytotoxic and proliferative responses against MM following dbBiTE arming. T cell proliferation was determined using CellTrace™ CFSE staining. We found that CD62L−CD45RA− T_{EM} cells induced significant CD107a expression and were positive for IFN-γ and TNF-α expression when co-cultured with MM.1S lines, whereas no cytotoxic activity was observed for CD62L⁺ CD45RA⁺ central memory (T_{CM}) T cells (FIGS. 5A-5B). Moreover, T_{EM} cells exhibited superior proliferative capacity in the presence of dbBiTE alone or dbBiTE with CS1 targets after 5 days of co-culture compared to T_{CM} cells (FIGS. 5C-5F). Interestingly, proliferating cells from the T_{EM} subset were mainly CD4 T cells but not CD8 T cells. Examination of dbBiTE-armed CD62L−CD45RA− PBMCs from MM patient samples also revealed significant CD107a (FIG. 5G) as well as IFN-γ expression following co-culture with the CS1 positive targets, MM.1S and U266B (FIG. 5H), highlighting the CS1-dbBiTE treatment against MM. Similar to T_{CM} cells, CD62L⁺ CD45RA⁺ stem cell memory (Tscm) T cells from healthy donors did not induce CD107a expression in the presence of MM.1S during co-culture. Thus, the data indicates that effector memory T cells are the dominant subsets for dbBiTE mediated effector function in patients and differential proportions of T_{EM} in patients contributes to varying responses to dbBiTE infusion clinically.

### Anti-Tumor Efficacy of CS1-dbBiTE Armed T Cells

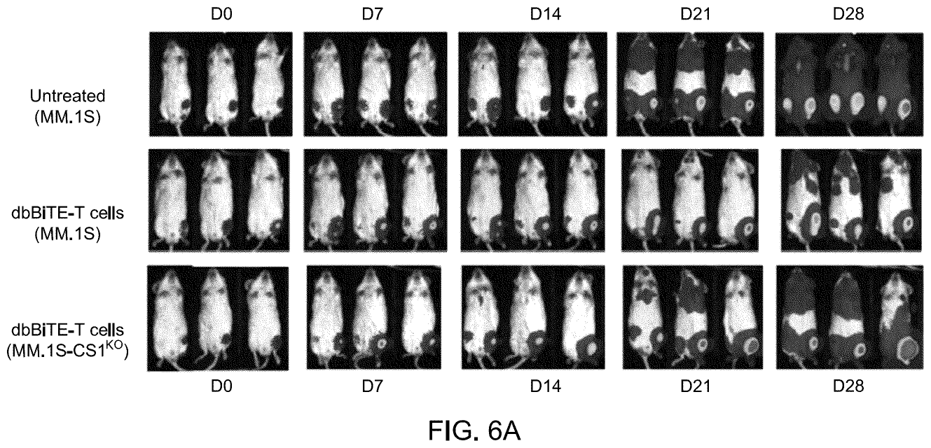

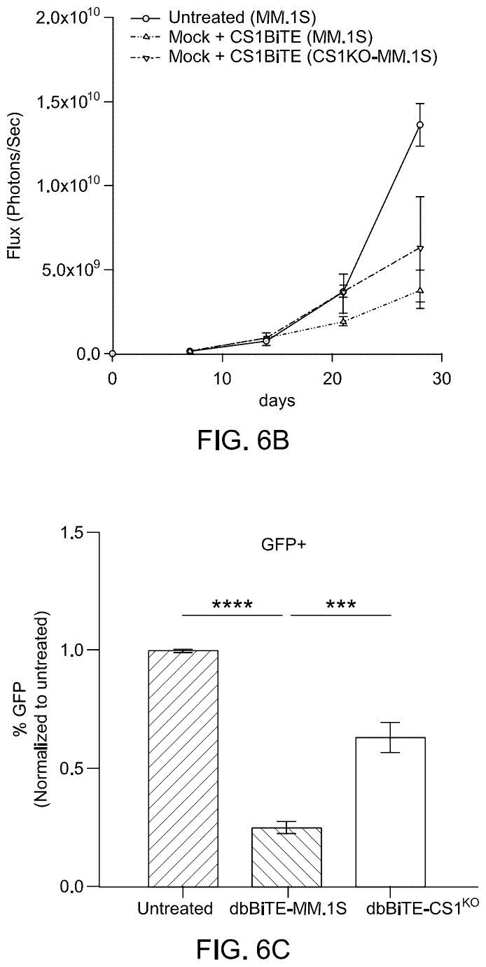

Given the demonstrated efficient cytotoxicity against MM, and cytokine production profile of dbBiTE armed T cells in vitro, the inventors hypothesized that armed T cells would possess superior anti-tumor efficacy against wildtype MM.1S tumor bearing mice over CS1-knockout MM xenograft mice. To this end, NSG mice were engrafted with 2×10⁶ MM.1S-eGFP or MM.1S-CS1^{KO}-eGFP lines via intra-tibial injections and administered PBS or 5×10⁶ dbBiTE-armed T cells intravenously, 6 days post tumor engraftment. Treatments were administered once weekly for 4 weeks, and tumor burden was monitored weekly by bioluminescence imaging for 5 weeks (FIG. 6A). MM.1S-eGFP xenograft mice that received dbBiTE-armed T cells were able to control tumor burden 4-5 weeks post treatment depicted by a reduction in flux compared to untreated mice and MM.1S-CS1^{KO} engrafted mice treated with CS1-dbBiTE (FIG. 6B). Examination of mice bone marrow (BM) samples also revealed similar patterns of residual tumor cells (GFP+), with untreated and MM.1S-CS1^{KO}-eGFP xenograft mice showing significantly higher GFP⁺ cells compared to MM.1S-eGFP engrafted mice (FIG. 6C). Although 5×10⁶ dbBiTE armed T cells were engrafted weekly into treatment groups, circulating T cells in mouse tissues (blood, spleen, and BM) post tissue harvest was not detected.

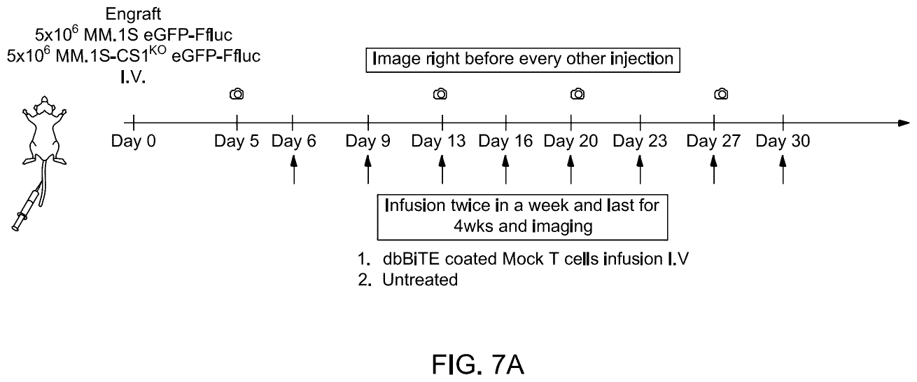

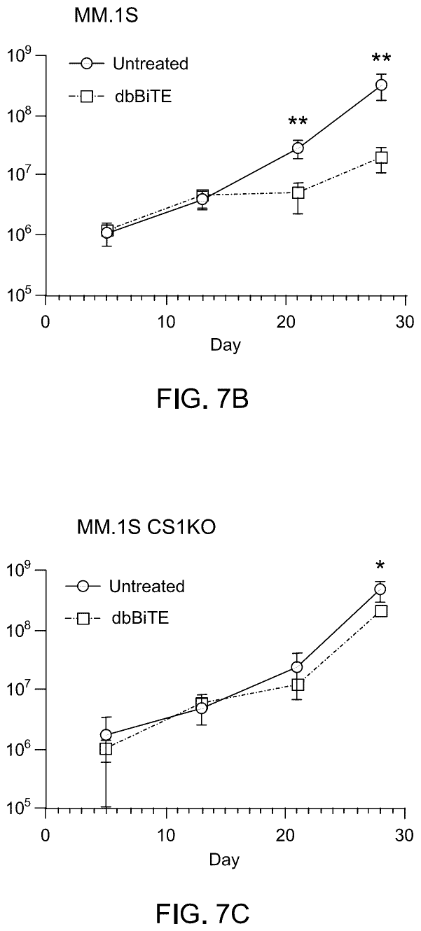

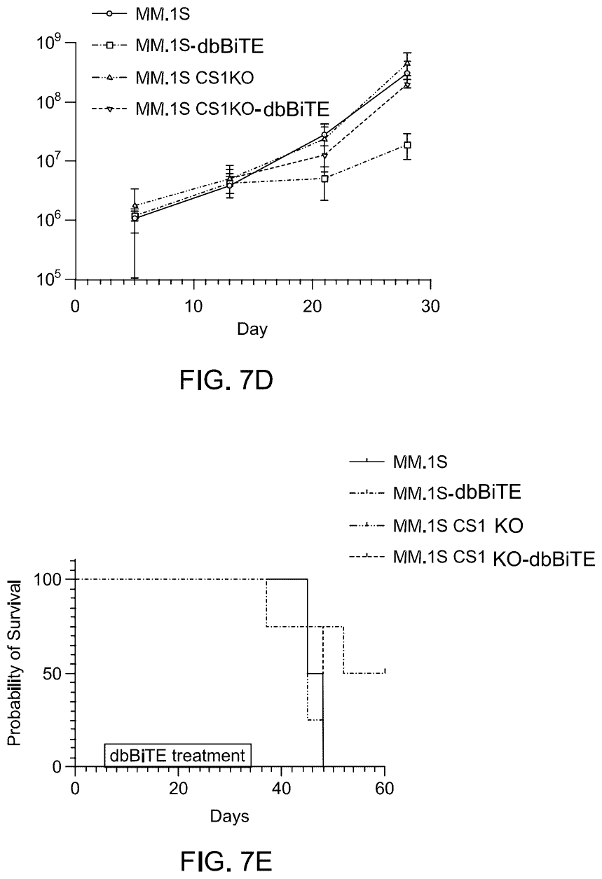

As shown in FIG. 7A, NSG mice were engrafted with 5×10⁶ MM.1S eGFP-Ffluc or 5×10⁶ CS1 KO MM.1S eGFP-Ffluc cells as controls. Subsequently, 5×10⁶ dbBiTE-coated (50 μg/ml) or uncoated T cells were injected into tumor-bearing mice on day 6 and twice a week for 4 weeks. Activated T cells were initially incubated in 1% goat serum in PBS for 30 minutes and subsequently coated with 50 ug/ml CS1 dbBiTE 30 minutes. Tumor progression in mice was monitored by bioluminescence imaging (BLI) following engraftment and weekly dbBiTE-T cell infusions. FIGS. 7B-7C provide a comparison of flux curves (photons/sec) between untreated and CS1-dbBiTE-T cell treated conditions, while FIG. 7D shows all data combined. The survival curves in FIG. 7E show that CS1-dbBiTE-coated T cells exhibited effective anti-tumor efficacy.

### DISCUSSION

In this study, the pre-clinical efficacy of human T cells armed with newly generated CS1-dbBiTEs was assessed against multiple myeloma and increased T cell activation and expansion was observed, as well as significant effector function including production of inflammatory cytokines and in vitro tumor lysis. Conversely, these observations were absent in unarmed T cells and significantly downregulated during co-culture with CS1 knockout MM targets. Traditionally, CD3-engaging bsAbs comprise two genetically-engineered single chain variable fragments (scFv), fused by a flexible linker. Alternatively, we cross-linked two intact antibodies to generate antibody molecules of dual specificities with bivalent binding (dbBiTE), circumventing the need to engineer bsAbs that may be difficult to properly fold or express, with poor pharmacokinetic properties when administered in patients.

Elotuzumab is an FDA-approved monoclonal antibody for treatment of MM, which exhibits a dual mechanism of action through direct NK cell activation when bound to SLAMF7 on its surface or by antibody dependent cellular cytotoxicity (ADCC). (Refs 24, 25). Additional modes of action have also been reported via other SLAMF7 positive cells including dendritic cells and CD8+ T cells. (Ref 26). Nevertheless, due to its suboptimal efficacy in patient trials, elotuzumab is used in combination with immunomodulatory drugs and steroids, such as lenalidomide and dexamethasone, to achieve better efficacy. (Refs 15, 27, 28). By conjugating intact elotuzumab and humanized anti-OKT3 antibodies, the inventors sought not only to improve the efficacy of elotuzumab but also benefit from its safety profile, which has been consistent across multiple clinical studies. Additionally, arming T cells ensured that the right binding affinity/biological activity of the fused molecule could be determined, allowing for removal of excess dbBiTEs. The relatively larger size of the dbBiTE (about 300 kDa) molecule is also likely to confer better pharmacokinetic properties in patients. In contrast, anti-CD3 antibodies such as OKT3 or UCHT1 antibodies can promote cross-linking of the TCR signaling complex leading to non-specific activation of T cells in the absence of antigen, ultimately resulting in substantial release of inflammatory cytokines and soluble mediators. (Refs. 29, 30). Indeed, IL-6 and TNF-α have been implicated as central mediators of cytokine release syndrome (CRS) a major toxicity associated with T cell immunotherapy. (Ref 31). For instance, clinical administration of an anti-CD28 antibody to healthy individuals in a phase one study resulted in peak levels of inflammatory cytokines TNF-α, IL-2, -6 and -10 as well as IFN-γ, and IL-1β within the first few hours of infusion. (Ref 32). In this study, arming T cells with db-BiTEs did not promote cytokine release in the absence of antigen suggesting the need for antigen dependency.

Conversely, clinical administration of T cells armed with bsAbs have been shown to lack indicators of CRS highlighting their safety. (Refs, 33, 34). In line with these studies, the inventors observed significantly lower IFN-γ or TNF-α positive cells as well as lower cytokine secretion from dbBiTE-armed T cells alone, with cytokine levels peaking only in the presence of antigen. Furthermore, considering the cell-based therapeutic approach, only microgram (μg) amounts of dbBiTEs are needed for T cell arming, thus limiting the occurrence of non-specific activation and cytokine release. In addition to utilizing FDA approved therapeutic antibodies for dbBiTE development, the use of μg concentrations can be achieved without further regulatory concerns. Such an approach could potentially provide ready-to-use antigen-specific T cells that may be used in combination with other cellular therapies such as CAR T cells for treatment of a variety of hematologic malignancies.

Although bsAbs and CAR T cells are at the forefront of cell-mediated cancer therapies, bsAbs possess numerous advantages over CAR T cells in the clinic, including immediate availability to patients. The short half-life also ensures that bsAb therapy can be halted at any time upon discovery of safety-related issues such as on target/off tumor effects. The inability to obtain sufficient T cells from some patients due to poor ‘fitness’ also implies that certain candidates remain ineligible for CAR T therapy but may still be able to obtain bsAb therapy, since it is likely they will maintain effector T cell populations with the capacity to respond rapidly to bsAb treatment. (Refs 35, 36). This data demonstrates that effector memory T cell populations, which likely require less stimulation to be fully activated, are the more responsive subsets (compared to central memory T cells) to cell proliferation and subsequent effector function when armed with CS1-dbBiTE. (Ref 23). The mechanism of bsAb tumor killing on the other hand, is reported to be similar to that of CAR T cells, involving initial synapse formation and subsequent release of soluble mediators such as perforins and granzymes. Nevertheless, given that bsAbs engage the natural CD3 complex, (whereas CARs do not) the synapse that forms between T cells and tumor targets when linked by bsAbs likely resembles synapses formed during physiologic T cell antigen recognition, leading to increased proliferation and upregulation of activation markers such as CD69 as demonstrated in this study. (Refs 37, 38). Therefore, although bsAbs are used by continually infusing into patients for better therapeutic efficacy, we find that dbBiTE-armed T cells also exhibit significantly potent effector function either in the presence or absence of in vitro bead activation, indicating that banked T cells (bead stimulated or not) can be obtained and armed with dbBiTE at any point when needed and dbBiTEs could be infused directly into patients to still achieve the desired clinical effect.

An advantage of CAR T over bsAb therapy, however, is the potential for CAR T cells to engraft long-term in vivo, providing a source of antigen-experienced T cells capable of responding to recurring tumors. (Refs 38, 39). In other studies, although bsAbs were administered in xenograft mice once daily for 5 days, the short half-life meant bsAbs were not present at later stages of tumor progression, even though it is likely to have delayed metastasis. (Ref 40). Similarly, studies in MM xenograft mice treated with novel BCMA/CD3 bsAbs after intraperitoneal transfer of ex-vivo expanded T cells also revealed that transferred T cells failed to engraft in non-responding mice following examination of tissues samples and were >98% positive for BCMA expression. (Ref 41). In line with these observations, we found that MM.1S xenograft mice receiving CS1-dbBiTE armed T cells (once weekly for 4 weeks) were able to reduce tumor burden compared to MM.1S-CS1^{KO} xenograft mice and untreated controls, however armed cells failed to engraft long term and were undetectable in mouse tissues (blood, spleen, and marrow) following analysis. Interestingly, we did not observe antigen loss in the relapsed tumor. However, it is worth noting that the same concentration of dbBiTE for both in vitro and in vivo experiments were used. It is reasonable to consider that a higher concentration of dbBiTE may be required to coat T cells for in vivo experiments to achieve better potency or more frequency of in vivo infusion instead of once a week. Considering the reduction in tumor burden observed, it is likely dbBiTE armed T cells may have reduced tumor metastasis and perhaps if followed longer, may have been able to prolong mouse survival.

In summary, this study has demonstrated an efficient approach for targeting MM tumors by utilizing intact and clinically available antibodies (elotuzumab and OKT3) fused to generate a bispecific molecule for redirecting T cells towards CS1-expressing MM killing. Although dbBiTEs are still in developmental stages, and applicable to a variety of therapeutic antibodies, our findings support the use of CS1-dbBiTEs as an effective therapeutic approach for targeting MM. Through the pre-complexed arming approach, dbBITE can be used in combination cellular therapy targeting different antigens. This approach offers the advantage of producing activated T cells as an additional side product during the manufacturing process, along with other cellular products derived from the same patients.

### Materials and Methods

Generation of CS1-dbBiTE. CS1 (2 mg, 13.33 nmol) in 200 μl of PBS was reduced with a 30-molar excess of tris (2-carboxyethyl) phosphine (TCEP) and 1 mM of EDTA at 37° C. for 2 h under Argon. The TCEP was removed by using a desalting spin column (Zeba, 7 KDa MW cutoff, Thermo Fisher Scientific, Waltham, MA). The reduced CS1 was reacted with a 20-fold molar excess of bromoacetamido-DBCO (Broadpharm, San Diego, CA) in pH 7.4 at RT overnight under Argon. The excess bromoacetamido-DBCO was removed by dialysis in PBS (2 L×5). The conjugation was confirmed by Agilent 6520 QTOF mass spectrometry. The light chain had one DBCO/light chain and the heavy chain an average of 3 DBCO/heavy chain. Anti-huor-OKT3 has been previously described²². Anti-hu-OKT-3 (5 mg, 33.33 nmol) in 858 μl of PBS was reduced with a 30-molar excess of tris (2-carboxyethyl) phosphine (TCEP) and 1 mM of EDTA at 37° C. for 2 h under Argon. The TCEP was removed by using a desalting spin column (Zeba, 7 KDa MW cutoff). The reduced anti-hu-OKT-3 was reacted with a 60-fold molar excess of bromoacetamide-PEG₅-N3 (Broadpharm, San Diego, CA) in pH 7.4 at RT overnight under Argon. The excess bromoacetamide-PEG₅-N3 was removed by dialysis in PBS (2 L×5). The conjugation was confirmed by Agilent 6520 QTOF mass spectrometry. The light chain had one PEG₅-N₃ and heavy chain an average of 3 PEG₅-N₃/heavy chain. CS1-DBCO (1.65 mg, 11.0 nmol) in 500 μl PBS was incubated with anti-hu-OKT3-PEG₅-N₃ (1.65 mg, 11.0 nmol) in 580 μl PBS, pH 7.4 at RT for 2 days under Argon. The clicked antibodies were purified by size exclusion chromatography on a Superdex 200, 10×300 GL column (GE Healthcare) at a flow rate of 0.5 ml/min in PBS using a GE AKTA Purifier. The 300 kDa peak was collected and concentrated to 2.93 mg/mL.

Antibodies and Flow Cytometry. Mouse monoclonal antibodies against human CD3, CD4, CD8, CD69, CD62L CD45RA, CD45RO, CD25, CD62L, CD107a were obtained from BD Biosciences. The anti-CS1 antibody was obtained from R&D Systems. Briefly, cells were harvested and washed twice in FSS solution (PBS buffer containing 2% FCS and 0.5% NAN₃). Staining with labeled antibodies was then carried out in the dark at 4° C. for 15 minutes according to manufacturer's protocol. Unless otherwise stated, antibodies were fluorochrome conjugated to APC, APC/Cy7, FITC, PE, or PE/Cy7, PerCP or Viogreen (Brilliant violet 510). Following staining, samples were washed twice with FSS solution before analysis. Cell viability was determined using Dapi staining. Nonreactive, isotype-matched antibodies were used as controls. Flow cytometry was carried out on MACSQuant (Miltenyi Biotec), and data analysis was performed with FCS Express Version 7 (De Novo Software) for Windows.

Cell lines. The multiple myeloma line MM.1S was purchased from ATCC and cultured in RPMI media supplemented with 10% heat inactivated FBS. To generate firefly luciferase⁺ GFP⁺ MM.1S (eGFP⁺ffluc⁺), MM.1S cells were transduced with lentiviral vector encoding eGFP-ffluc; transduced cells were further sorted by FACS to obtain >98% purity. MM.1S-CS1^{KO} cells were generated by CRISPR/Cas9 system using appropriate guide RNAs (gRNA) against SLAMF7, and knockout population were sorted using anti-CS1 antibody (R&D Systems). (Ref 42). Prior to cryopreservation, cell lines were authenticated for expression (or lack) of desired surface markers by flow cytometry and thawed cells were cultured for 3-6 weeks prior to use in assays.

T cell isolation and activation. Leukapheresis products were obtained from healthy donors after obtaining informed consent, and peripheral blood mononuclear cells (PBMCs) were separated by density gradient centrifugation on Ficoll-paque (Amersham Biosciences). T naïve/memory (Tn/mem) cells were isolated by autoMACS (Miltenyi Biotec) using CD62L⁺ magnetic beads following depletion of CD14⁺ and CD25⁺ cell fractions, and resulting cells were activated with CD3/CD28 microbeads (Invitrogen) as previously described in Refs 43 and 44. In some experiments, healthy donor PBMCs were FACS sorted for central memory (Tcm), effector memory (Tem) and stem cell memory (Tscm) T cells without CD3/CD28 bead stimulation. All healthy donor samples were obtained under approved City of Hope Institutional Review Board (COH IRB) protocols (IRB09025) in accordance with the Declaration of Helsinki.

Coating conditions for CS1-dbBiTE. Target cells (MM.1S and activated CD3⁺ Tn/mem cells) were initially incubated in 1% goat serum in PBS for 30 minutes and subsequently incubated on ice with increasing concentrations of CS1-dbBiTE antibody (0.1 ug/mL to 50 ug/mL per 10×10⁶ cells) for 15-30 minutes. In some experiments, incubation was carried out for 1 hour. Cells were then washed twice to eliminate excess unbound dbBiTE and labelled with 2 ug/mL goat anti-mouse Alexa647 or goat anti-human Alexa555 secondary antibodies (Thermofisher Scientific) for detection of bound CS1-dbBiTE. Following wash steps, cells were resuspended in PBS and analyzed by flow cytometry on MACSQuant (Miltenyi). Unlabeled dbBiTE cells or cells labeled with secondary antibodies only were used as controls. In co-culture experiments, T cells (in complete RPMI media) were armed with dbBiTE for 15 minutes at room temperature, washed twice with plain RPMI media before use in assay.

Degranulation assay. Degranulation assay was done as previously described in Ref 43. Briefly, CS1dbBiTE-armed T cells or PBMCs were co-cultured with MM.1S and MM.1S-CS1^{KO} target lines at an effector to target (e:t) ratio of 2:1 in complete RPMI medium containing Golgi Stop solution (BD Biosciences). Anti-CD107a antibody was added to each well and the co-culture setup was incubated for 5-6 hours at 37° C. Unarmed T cells were used as controls. Degranulation was assessed by multicolor flow cytometry on MACSQuant analyzer (Miltenyi).

Intracellular cytokine staining. Target cells (0.2×10⁶) were plated in 96-well plates and co-cultured with 0.2×10⁶ CS1dbBiTE-armed CD3⁺ T cells for 4 hours at 37° C. in RPMI media, supplemented with 10% FBS. Golgi plug media (brefeldin A; BD Biosciences) was subsequently added to each well and incubated overnight at 37° C. Cells were washed and stained for surface marker detection prior to intracellular staining. Following fixation and permeabilization, (cytoperm kit; BD Biosciences), cells were stained with anti-IFNγ and anti-TNFα antibodies (BD Biosciences) for 30 minutes at 4° C. and washed twice with perm wash (BD Biosciences) before analysis on MACSQuant analyzer (Miltenyi). Unarmed T cells were used as control.

Cytokine analysis. CS1-dbBiTE armed T cells were co-cultured at 1:1 e:t ratio with MM.1S or MM.1S-CS1^{KO} target cells overnight in 96-well plates. The culture supernatant was collected and stored at −20° C. until analysis. Cytokines were measured with the Human Cytokine Magnetic Bead Array (10-plex), according to the manufacturer's instructions (Invitrogen).

In vitro cytotoxicity assay. Cytotoxicity assay was carried out as previously described. Briefly, eGFP-ffluc-expressing MM.1S and MM.1S-CS1^{KO} target cells were plated in 96-well plates at a concentration of 0.1×10⁶ per well in 100 L of RPMI media containing 10% FBS. CS1-dbBiTE-armed, resting and activated T cells were then added at varying concentrations to final e:t ratios of 0.25:1, 0.5:1, 1:1, 2.5:1, 5:1 and 10:1, respectively. In both conditions, unarmed T cells were used as controls and plated according to same e:t ratios. Co-culture cells were incubated for 24 hours at 37° C. and analyzed by flow cytometry. Tumor killing was determined by percentage expression of eGFP-ffluc⁺ cells.

CFSE proliferation assay. Tumor cells (0.1×10⁶) were co-cultured in RPMI medium for 96 hours with CS1-dbBiTE armed, central (CD62L⁺ CD45RA⁺) and effector memory (CD62L−CD45RA−) T cells at 1:1 e:t ratio. Prior to co-culture, armed T cells were labelled with or without 5 μM CellTrace CFSE (Thermo Fisher Scientific) and incubated for 20 minutes at 37° C. Thereafter, cells were harvested and stained with mouse anti-human CD3, CD4 and CD8 antibodies before analysis. T cell proliferation by the different subsets was determined by flow cytometry on MACSQuant.

Xenograft models. All mouse experiments were approved by the City of Hope Institutional Animal Care and Use Committee and were performed in NOD.Cg-Prkdcscid Il2rgtm1Wjl/SzJ mice (NSG; 6-10 weeks old). Mice were kept in pie cages in a specific pathogen free (SPF) room, with a maximum of 5 mice per cage. NOD/SCID IL2RgCnull (NSG) mice were engrafted on day 0 with 2×10⁶ eGFP⁺ffluc⁺ MM.1S or MM.1S-CS1^{KO} target cells via intra-tibial delivery (i.t). Mice were imaged and sorted on day five following tumor engraftment, and subsequently treated next day with 5×10⁶ unarmed or CS1-dbBiTE-armed T cells intravenously (i.v). Infusions with armed or unarmed T cells (5×10⁶/mouse) were carried out once a week for 4 weeks. Effector T cells were armed with 1 ug/ml CS1-dbBiTE per 10×10⁶ cells. Tumor burden was monitored once weekly by bioluminescence imaging using Xenogen imager (Spectral Instruments Imaging). Mice were sacrificed after 5 weeks post-tumor engraftment and tissue samples were harvested for analysis by flow cytometry. Mice euthanasia was carried out using carbon dioxide (CO₂) in an euthanasia chamber before cervical dislocation and tissue collection.

Statistical analysis. Data was analyzed using GraphPad Prism version 9 for Windows (GraphPad Software, San Diego, CA), and values expressed as mean±SD from independent experiments unless otherwise stated. Paired t tests or ordinary one-way ANOVA was used when comparing between two groups or three or more groups, respectively. For animal studies, Kaplan-Meier survival analysis was performed, and statistical significance was calculated using log-rank (Mantel-Cox). A p value ≤0.05 was considered statistically significant.

The examples are also described in Awuah et al, Scientific Reports, Volume 13, Article Number 20853, published on-line Nov. 27, 2023, the disclosure of which is incorporated by reference herein in its entirety. The Supplemental Information to this article, including Supplementary FIGS. 1-4, is incorporated by reference herein in its entirety.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Informal Sequence Listing
Light Chain Variable Domain of humanized
OKT3 antibody
=CDR L1:
SEQ ID NO: 1
SASSSVSYMN
=CDR L2:
SEQ ID NO: 2
DTSKLA
=CDR L3:
SEQ ID NO: 3
QQWSSNPFT
(Signal Sequence:) =
SEQ ID NO: 7
AATMETDTLLLWVLLLWVPGSTG
(Light Chain Variable Domain)
SEQ ID NO: 9
DIQMTQSPSSLSASVGDRVTITCSASSSVSYMNWYQQTPGKAPKRWIYDT
SKLASGVPSRFSGSGSGTDYTFTISSLQPEDIATYYCQQWSSNPFTFGQG
TKLQITR
Heavy Chain Variable Domain of humanized
OKT3 antibody
=CDR H1:
SEQ ID NO: 4
RYTMH
=CDR H2:
SEQ ID NO: 5
YINPSRGYTNYNQKVKD
=CDR H3:
SEQ ID NO: 6
YYDDHYCL
(Signal Sequence) =
SEQ ID NO: 8
LAATMKCSWVIFFLMAVVTGVNSQV
(Heavy Chain Variable Domain)
SEQ ID NO: 10
QLVQSGGGVVQPGRSLRLSCKASGYTFTRYTMHWVRQAPGKGLEWIGYIN
PSRGYTNYNQKVKDRFTISTDKSKSTAFLQMDSLRPEDTAVYYCARYYDD
HYCLDYWGQGTPVTV
SEQ ID NO: 11
ANESHNGSILPISWRWGESDMTFICVARNPVSRNFSSPILARKLCEGAAD
DPDSSMV
=Elotuzumab Light Chain
SEQ ID NO: 12
DIQMTQSPSSLSASVGDRVTITCKASQDVGIAVAWYQQKPGKVPKLLIYW
ASTRHTGVPDRFSGSGSGTDFTLTISSLQPEDVATYYCQQYSSYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
=Elotuzumab Heavy Chain
SEQ ID NO: 13
EVQLVESGGGLVQPGGSLRLSCAASGFDFSRYWMSWVRQAPGKGLEWIGE
INPDSSTINYAPSLKDKFIISRDNAKNSLYLQMNSLRAEDTAVYYCARPD
GNYWYFDVWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Elotuzumab Light Chain Variable Domain
SEQ ID NO: 14
DIQMTQSPSSLSASVGDRVTITCKASQDVGIAVAWYQQKPGKVPKLLIYW
ASTRHTGVPDRFSGSGSGTDFTLTISSLQPEDVATYYCQQYSSYPYTFGQ
GTKVEIK
=CDR L1:
SEQ ID NO: 15
KASQDVGIAVA
=CDR L2:
SEQ ID NO: 16
WASTRHT
=CDR L3:
SEQ ID NO: 17
QQYSSYPYT
Elotuzumab Heavy Chain Variable Domain
SEQ ID NO: 18
EVQLVESGGGLVQPGGSLRLSCAASGFDFSRYWMSWVRQAPGKGLEWIGE
INPDSSTINYAPSLKDKFIISRDNAKNSLYLQMNSLRAEDTAVYYCARPD
GNYWYFDVWGQGTLVTVSS
=CDR H1:
SEQ ID NO: 19
RYWMS
=CDR H2:
SEQ ID NO: 20
EINPDSSTINYAPSLKD
=CDR H3:
SEQ ID NO: 21
PDGNYWYFDV
SEQ ID NO: 22
AATMETDTLLLWVLLLWVPGSTGDIQMTQSPSSLSASVGDRVTITCSASS
SVSYMNWYQQTPGKAPKRWIYDTSKLASGVPSRFSGSGSGTDYTFTISSL
QPEDIATYYCQQWSSNPFTFGQGTKLQITR
SEQ ID NO: 23
LAATMKCSWVIFFLMAVVTGVNSQVQLVQSGGGVVQPGRSLRLSCKASGY
TFTRYTMHWVRQAPGKGLEWIGYINPSRGYTNYNQKVKDRFTISTDKSKS
TAFLQMDSLRPEDTAVYYCARYYDDHYCLDYWGQGTPVTV

### REFERENCES

1 Siegel et al C A Cancer J Clin 70, 7-30 (2020). https://doi.org:10.3322/caac.21590
2 Kodama et al. Anti-GPRC5D/CD3 Bispecific T-Cell-Redirecting Antibody for the Treatment of Multiple Myeloma. Mol Cancer Ther 18, 1555-1564 (2019).
3 Verkleij et al. Preclinical activity and determinants of response of the GPRC5DxCD3 bispecific antibody talquetamab in multiple myeloma. Blood Adv 5, 2196-2215 (2021).
4 von Stackelberg, A. et al. J Clin Oncol 34, 4381-4389 (2016).
5 Zuch de Zafra, C. L. et al. Targeting Multiple Myeloma with AMG 424, a Novel Anti-CD38/CD3 Bispecific T-cell-recruiting Antibody Optimized for Cytotoxicity and Cytokine Release. Clin Cancer Res 25, 3921-3933 (2019).
6 Kontermann, R. E. & Brinkmann, U. Bispecific antibodies. Drug Discovery Today 20, 838-847 (2015). https://doi.org:https://doi.org/10.1016/j.drudis.2015.02.008
7 Wu, Z. & Cheung, N. V T cell engaging bispecific antibody (T-BsAb): From technology to therapeutics. Pharmacology & therapeutics 182, 161-175 (2018).
8 Subklewe, M. BiTEs better than CAR T cells. Blood Advances 5, 607-612 (2021).
9 Wong et al, Haematologica 98, 1930-1938 (2013).
10 Chu, J. et al. CS1-specific chimeric antigen receptor (CAR)-engineered natural killer cells enhance in vitro and in vivo antitumor activity against human multiple myeloma. Leukemia 28, 917-927 (2014). https://doi.org:10.1038/leu.2013.279
11 Hsi, E. D. et al. CS1, a potential new therapeutic antibody target for the treatment of multiple myeloma. Clin Cancer Res 14, 2775-2784 (2008).
12 Rosenzweig, M. et al. Preclinical data support leveraging CS1 chimeric antigen receptor T-cell therapy for systemic light chain amyloidosis. Cytotherapy 19, 861-866 (2017).
13 Tai, Y T. et al. Anti-CS1 humanized monoclonal antibody HuLuc63 inhibits myeloma cell adhesion and induces antibody-dependent cellular cytotoxicity in the bone marrow milieu. Blood 112, 1329-1337 (2008). https://doi.org:10.1182/blood-2007-08-107292
14 Dimopoulos et al. Blood Cancer Journal 10, 91 (2020).
15 Richardson et al. Lancet Haematol 2, e516-527 (2015).
16 Wang, X. et al. Clinical cancer research: an official journal of the American Association for Cancer Research 24, 106-119 (2018).
17 Ali, S. A. et al. T cells expressing an anti-B-cell maturation antigen chimeric antigen receptor cause remissions of multiple myeloma. Blood 128, 1688-1700 (2016).
18 Brudno, J. N. et al. T Cells Genetically Modified to Express an Anti-B-Cell Maturation Antigen Chimeric Antigen Receptor Cause Remissions of Poor-Prognosis Relapsed Multiple Myeloma. J Clin Oncol 36, 2267-2280 (2018). https://doi.org:10.1200/JCO.2018.77.8084
19 Raje, N. et al. Anti-BCMA CAR T-Cell Therapy bb2121 in Relapsed or Refractory Multiple Myeloma. N Engl J Med 380, 1726-1737 (2019). https://doi.org:10.1056/NEJMoa1817226
20 Caraccio et al, Bispecific Antibodies for Multiple Myeloma: A Review of Targets, Drugs, Clinical Trials, and Future Directions. Frontiers in immunology 11, 501-501 (2020).
21 Lum et al, Anti-CS1×Anti-CD3 Bispecific Antibody (BiAb)-Armed Anti-CD3 Activated T Cells (CS1-BATs) Kill CS1+ Myeloma Cells and Release Type-1 Cytokines. Frontiers in Oncology 10 (2020). https://doi.org:10.3389/fonc.2020.00544
22 Kujawski, M. et al. Generation of dual specific bivalent BiTEs (dbBIspecific T-cell engaging antibodies) for cellular immunotherapy. BMC cancer 19, 882-882 (2019).
23 Bargou, R. et al. Tumor Regression in Cancer Patients by Very Low Doses of a T Cell-Engaging Antibody. Science 321, 974 (2008). https://doi.org:10.1126/science.1158545
24 Collins, S. M. et al. Elotuzumab directly enhances NK cell cytotoxicity against myeloma via CS1 ligation: evidence for augmented NK cell function complementing ADCC. Cancer Immunol Immunother 62, 1841-1849 (2013). https://doi. org:10.1007/s00262-013-1493-8
25 Lonial, S. et al. Elotuzumab Therapy for Relapsed or Refractory Multiple Myeloma. New England Journal of Medicine 373, 621-631 (2015). https://doi.org:10.1056/NEJMoa1505654
26 Ritchie et al, 11, 261-266 (2018). https://doi.org:10.1111/cts.12532
27 Dimopoulos et al. New England Journal of Medicine 379, 1811-1822 (2018).
28 Goldschmidt et al. Blood 138, 486-486 (2021). https://doi.org:10.1182/blood-2021-147323
29 Ellerman, D. Bispecific T-cell engagers: Towards understanding variables influencing the in vitro potency and tumor selectivity and their modulation to enhance their efficacy and safety. Methods 154, 102-117 (2019). https://doi.org:https://doi.org/10.1016/j.ymeth.2018.10.026
30 Jang, S. et al. Development of an antibody-like T-cell engager based on VH-VL heterodimer formation and its application in cancer therapy. Biomaterials 271, 120760 (2021).
31 Park et al, Potent ex vivo armed T cells using recombinant bispecific antibodies for adoptive immunotherapy with reduced cytokine release. J Immunother Cancer 9 (2021).
32 Suntharalingam et al. N Engl J Med 355, 1018-1028 (2006).
33 Lum, L. G. et al. Multiple infusions of CD20-targeted T cells and low-dose IL-2 after SCT for high-risk non-Hodgkin's lymphoma: a pilot study. Bone Marrow Transplant 49, 73-79 (2014).
34 Vaishampayan, U., Thakur, A., Rathore, R., Kouttab, N. & Lum, L. G. Phase I Study of Anti-CD3×Anti-Her2 Bispecific Antibody in Metastatic Castrate Resistant Prostate Cancer Patients. Prostate Cancer 2015, 285193 (2015). https://doi.org:10.1155/2015/285193
35 DiLillo, D. J. et al. A BCMAxCD3 bispecific T cell-engaging antibody demonstrates robust antitumor efficacy similar to that of anti-BCMA CAR T cells. Blood Adv 5, 1291-1304 (2021). https://doi.org:10.1182/bloodadvances.2020002736
36 Graham et al, Cells 7 (2018). https://doi.org:10.3390/cells7100155
37 Brischwein, K. et al. Strictly Target Cell-dependent Activation of T Cells by Bispecific Single-chain Antibody Constructs of the BiTE Class. Journal of Immunotherapy 30 (2007).
38 Slaney, C. Y, Wang, P., Darcy, P. K. & Kershaw, M. H. CARs versus BiTEs: A Comparison between T Cell-Redirection Strategies for Cancer Treatment. Cancer Discov 8, 924-934 (2018).
39 Slaney, C. Y et al. Dual-specific Chimeric Antigen Receptor T Cells and an Indirect Vaccine Eradicate a Variety of Large Solid Tumors in an Immunocompetent, Self-antigen Setting. Clin Cancer Res 23, 2478-2490 (2017). https://doi.org:10.1158/1078-0432.Ccr-16-1860
40 Horn, L. A. et al. CD3×PDL1 bi-specific T cell engager (BiTE) simultaneously activates T cells and NKT cells, kills PDL1(+) tumor cells, and extends the survival of tumor-bearing humanized mice. Oncotarget 8, 57964-57980 (2017). https://doi.org:10.18632/oncotarget.19865
41 Hipp, S. et al. A novel BCMA/CD3 bispecific T-cell engager for the treatment of multiple myeloma induces selective lysis in vitro and in vivo. Leukemia 31, 1743-1751 (2017).
42 Zah, E. et al. Systematically optimized BCMA/CS1 bispecific CAR-T cells robustly control heterogeneous multiple myeloma. Nature Communications 11, 2283 (2020).
43 Urak, R. et al. Journal for immunotherapy of cancer 5, 26-26 (2017).
44 Wang, X. et al. Blood 118, 1255-1263 (2011)

What is claimed is:

1. A compound comprising an anti-CD3 antibody covalently bound to a CS-1-binding antibody.

2. The compound of claim 1, wherein the anti-CD3 antibody comprises an OKT3 antibody and the CS-1-binding antibody comprises elotuzumab.

3. The compound of claim 2, wherein the anti-CD3 antibody comprises:
(i) a light chain variable domain CDR 1 as set forth in SEQ ID NO:1, CDR 2 as set forth in SEQ ID NO:2 and CDR 3 as set forth in SEQ ID NO:3; and
(ii) a heavy chain variable domain CDR 1 as set forth in SEQ ID NO:4, CDR 2 as set forth in SEQ ID NO:5 and CDR 3 as set forth in SEQ ID NO:6.

4. The compound of claim 3, wherein anti-CD3 antibody comprises:
(i) a light chain variable domain having at least 95% sequence identity to SEQ ID NO:9; and
(ii) a heavy chain variable domain having at least 95% sequence identity to SEQ ID NO:10.

5. The compound of claim 4, wherein the anti-CD3 antibody comprises:
(i) a light chain variable domain having SEQ ID NO:9; and
(ii) a heavy chain variable domain having SEQ ID NO:10.

6. The compound of claim 4, wherein:
(i) the 5′ end of the light chain variable domain further comprises a signal sequence as set forth in SEQ ID NO:7; and
(ii) the 5′ end of the heavy chain variable domain further comprises a signal sequence as set forth in SEQ ID NO:8.

7. The compound of claim 2, wherein the CS-1-binding antibody comprises:
(i) a light chain variable domain CDR L1 as set forth in SEQ ID NO:15, CDR L2 as set forth in SEQ ID NO:16, and CDR L3 as set forth in SEQ ID NO:17; and
(ii) a heavy chain variable domain CDR H1 as set forth in SEQ ID NO:19, CDR H2 as set forth in SEQ ID NO:20, and CDR H3 as set forth in SEQ ID NO:21.

8. The compound of claim 2, wherein the CS-1-binding antibody comprises:
(i) a light chain variable domain having at least 95% sequence identity to SEQ ID NO:14; and
(ii) a heavy chain variable domain having at least 95% sequence identity to SEQ ID NO:18.

9. The compound of claim 8, wherein the CS-1-binding antibody comprises:
(i) a light chain variable domain having SEQ ID NO:14; and
(ii) a heavy chain variable domain having SEQ ID NO:18.

10. The compound of claim 8, wherein the CS-1-binding antibody comprises a light chain having at least 95% sequence identity to SEQ ID NO:12 and a heavy chain having at least 95% sequence identity to SEQ ID NO:13.

11. The compound of claim 10, wherein the CS-1-binding antibody comprises a light chain having SEQ ID NO:12 and a heavy chain having SEQ ID NO:13.

12. The compound of claim 1, wherein the CS-1-binding antibody comprises SEQ ID NO:11.

13. The compound of claim 1, wherein the anti-CD3 antibody is an antibody, a F(ab′) fragment, a F(ab′)₂ fragment, or a single chain variable domain; and wherein the CS-1-binding antibody is an antibody, a F(ab′) fragment, a F(ab′)₂ fragment, or a single chain variable domain.

14. The compound of claim 1, wherein a cysteine residue in the anti-CD3 antibody is covalently bound to a cysteine residue in the CS-1-binding antibody.

15. The compound of claim 1, wherein a cysteine residue in the hinge region of the anti-CD3 antibody is covalently bound to a cysteine residue in the hinge region of the CS-1-binding antibody.

16. The compound of claim 1, wherein a cysteine residue in the anti-CD3 antibody is covalently bound to a cysteine residue in the CS-1-binding antibody via a linking group of the formula: -L¹-L²-L³-, wherein L¹, L², and L³ are each independently a bond, an amino acid, a peptide, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

17. The compound of claim 1, wherein a cysteine residue in the hinge region of the anti-CD3 antibody is covalently bound to a cysteine residue in the hinge region of the CS-1-binding antibody via a linking group of the formula: -L¹-L²-L³-, wherein L¹ is a 2 to 12 membered substituted or unsubstituted heteroalkylene; L² is a substituted or unsubstituted fused heteroarylene having from 2 to 4 rings fused together; and L³ is —W(OCH₂CH₂)ₙ where W is a bond or unsubstituted C₁₋₆ alkylene and n is an integer from 1 to 100; wherein L¹ is covalently bound to the cysteine residue in the hinge region of the anti-CD3 antibody and L³ is covalently bound to the cysteine residue in the hinge region of the CS-1-binding antibody.

18. The compound of claim 1, wherein the compound is non-covalently bound to an immune cell, wherein the immune cell is a T cell, a B cell, a natural killer cell, a monocyte, a neutrophil, a macrophage, or a genetically-engineered immune cell.

19. A composition comprising the compound of claim 1 and
(i) a pharmaceutically acceptable excipient;
(ii) an immune cell selected from the group consisting of a T cell, a B cell, a natural killer cell, a monocyte, a neutrophil, a macrophage, or a genetically-engineered immune cell; or
(iii) a pharmaceutically acceptable excipient and an immune cell selected from the group consisting of a T cell, a B cell, a natural killer cell, a monocyte, a neutrophil, a macrophage, or a genetically-engineered immune cell.

20. A method of treating multiple myeloma in a patient in need thereof, the method comprising administering to the patient an effective amount of the compound of claim 1, thereby treating multiple myeloma in the patient.
